(19) United States
(12) Patent Application Publication
(10) Pub. No.: US 2023/0277524 A1
(43) Pub. Date: Sep. 7, 2023

(54) COMBINATION THERAPY FOR TREATMENT OF VIRAL INFECTIONS

(71) Applicant: Trailhead Biosystems Inc., Cleveland, OH (US)

(72) Inventors: Jan JENSEN, Shaker Heights, OH (US); Div TRIVEDI, Cleveland Heights, OH (US)

(21) Appl. No.: 17/925,783

(22) PCT Filed: May 21, 2021
(86) PCT No.: PCT/US2021/033689

§ 371 Date: Nov. 16, 2022

Related U.S. Application Data
(60) Provisional application No. 63/071,848, filed on Aug. 28, 2020.
(60) Provisional application No. 63/057,829, filed on Jul. 28, 2020.
(60) Provisional application No. 63/028,844, filed on May 22, 2020.

(51) Int. Cl. A61K 31/473; A61K 31/706; A61K 31/122; A61P 31/14

(52) U.S. Cl. CPC A61K 31/473; A61K 31/706; A61K 31/122; A61P 31/14

(57) ABSTRACT

The present invention relates to combination therapies for treating viral infections, particularly infections by severe acute respiratory syndrome corona virus 2 (SARS-CoV-2, also known as coronavirus disease 2019 (COVID-19).

### CROSS REFERENCE TO RELATED APPLICATIONS

This application claims priority to U.S. Provisional Patent Application Nos. 63/028,844 (filed May 22, 2020), 63/057,829 (filed Jul. 28, 2020), and 63/071,848 (filed Aug. 28, 2020) the contents of which are hereby incorporated by reference.

### BACKGROUND

New infectious diseases are constantly emerging, including those caused by viruses. For example, three new coronaviruses have emerged from animal reservoirs over the past two decades to cause serious and widespread illness and death. Currently, the world is experiencing an unprecedented pandemic. Coronavirus disease 2019 (COVID-19) is an infectious disease caused by severe acute respiratory syndrome coronavirus 2 (SARS-CoV-2). In the United States, more than 1.5 million people have been diagnosed with COVID-19, and over 90,000 cases have been fatal. Accordingly, there is an urgent need for antiviral therapies, such as COVID-19 therapies, particularly those involving drugs which already have been shown to be safe in humans and therefore can be quickly approved.

Rapid development of interventions that may provide clinical efficacy against emerging viral pandemics is relevant, and in the case of SARS-CoV2 critical. To this end, multiple avenues of research have been explored, including extensive machine-based bioinformatics-driven approaches, high-throughput drug screening, re-purposing of existing drugs, and accelerated development of vaccines. While increasingly powered by the rapid growth of data, bioinformatics-based approaches need to be complemented by empirical testing, as hits emerge through prediction-based scoring. High-throughput drug screening is a powerful method to identify novel drugs, especially when a target is identified, but its utility is less effective when the goal is interference with the lifecycle of a virus, such as SARS-CoV2. Singular drugs that have proven highly effective against other viruses are rare. Progress in vaccine development is rapid, and record-breaking, yet it remains unknown if vaccination against SARS-CoV2 may provide lasting immunity. Accordingly, improved anti-viral combinatorial therapies against SARS-CoV2 are needed.

### SUMMARY OF THE INVENTION

Provided herein are methods of treating a subject infected with a virus, e.g., severe acute respiratory syndrome coronavirus 2 (SARS-CoV-2) or related viruses (e.g., a virus of the family coronaviridae, arenaviridae, and/or filoviridae) by administering an effective amount of a particular combination of compounds which synergistically inhibits the virus and thus treat the infection.

In one embodiment, the combination comprises remdesivir, or analog thereof, and at least one ABCB1 channel transport inhibitor, preferably a dual-specificity ABCB1/ABCG2 inhibitor (Glycoprotein-1). In a particular embodiment, the ABCB1/ABCG2 inhibitor is elacridar, tariquidar, zosuquidar, or analogs thereof. In another embodiment, the combination further comprises an antioxidant. In a particular embodiment, the antioxidant is a Nuclear factor erythroid 2-related factor 2 (NRF2) agonist. In another particular embodiment, the NRF2 agonist is curcumin (also known as diferuloylmethane), or analog thereof,

Accordingly, as described herein, the invention relates to a particular combination of agents (also referred to herein as a “REC combination” or “REC compound”) which synergistically exhibits significantly improved potent anti-viral activity compared to previously known antiviral compounds. As a result, the combinations or compositions of the invention can be administered at significantly lower dosages than previous anti-viral compounds, including oral dosages. In a particular embodiment, the combination comprises remdesivir and elacridar, optionally in combination with curcumin, as well as analogues and functional equivalent variants thereof. In another particular embodiment, the combination comprises remdesivir and tariquidar, optionally in combination with curcumin, as well as analogues and functional equivalent variants thereof. Such combinations can be administered simultaneously (e.g., in a single formulation or concurrently as separate formulations). Alternatively, in another embodiment, the combinations are administered sequentially (e.g., as separate formulations).

Also provided are kits that include the combinations or compositions of the invention in a therapeutically effective amount adapted for use in the methods described herein.

### BRIEF DESCRIPTION OF THE DRAWINGS

FIG. 1 is a graph showing antiviral response data for the corresponding Tox and Viral response at 500 nM.

FIG. 2 is a graph comparing the inhibitory activity of antiviral compounds.

FIG. 3 is a graph comparing the inhibitory activity of antiviral compounds.

FIG. 4 is a graph comparing the inhibitory activity of antiviral compounds.

FIG. 5 is a graph comparing the inhibitory activity of antiviral compounds.

FIG. 6 shows a plot of the system response for the condition of added remdesivir (R), elacridar (E) and curcumin (C) top row, P3 (plate 3), VIRUS, factors provided each at 250 nM concentration final (X-axis lists stock concentrations in experiments).

FIG. 7 shows a plot of the system response for the condition of added remdesivir (R), elacridar (E) and curcumin (C) top row, P3 (plate 3), VIRUS, factors provided each at 250 nM concentration final (X-axis lists stock concentrations in experiments).

FIG. 8 shows a plot of the system response for the condition of added remdesivir (R), elacridar (E) and curcumin (C) top row, P3 (plate 3), VIRUS, factors provided each at 250 nM concentration final (X-axis lists stock concentrations in experiments).

FIG. 9 sows a plot of the system response for the condition of added remdesivir (R), elacridar (E) and curcumin (C) top row, P3 (plate 3), VIRUS, factors provided each at 250 nM concentration final (X-axis lists stock concentrations in experiments).

FIG. 10 is a coefficient plot showing toxicity correlates to loss of fluorescence intensity.

FIG. 11 is a coefficient plot of Plate 21, focusing on the REC combination at 250 nM, which shows that the ABCG2 inhibitory compounds FEBUX (febuxostat) and Ko143 do not substitute for ELACRIDAR (Elac).

FIG. 12 is a coefficient plot of Plate 23, focusing on the REC combination at 250 nM.

FIG. 13 is a coefficient plot of Plate 24, focusing on the REC combination at 250 nM.

FIG. 14 shows the system optimization of Plate 24.

FIG. 15 is a coefficient plot of Plate 22, focusing on the REC combination at 250 nM.

FIG. 16 shows the system optimization of Plate 22.

FIG. 17 is a coefficient plot of Plate 26.

FIG. 18 shows the system optimization of Plate 26.

FIGS. 19A and 19B are dose-response analyses of the REC combination.

FIG. 20 is a chart showing the eight subfamilies of ABC genes.

FIG. 21 is a graph comparing the results of a cell-based antiviral assay with SARS-CoV-2 in Huh7 cells.

FIGS. 22A and 22B are graphs comparing the results of a cell-based antiviral assay with SARS-CoV-2 in Vero E6 WT cells (A) and Vero E6 GFP cells (B).

FIGS. 23A and 23B are plots of the levels of viral genome copies (A) and infectious virus (B) in lungs of Syrian golden hamsters.

### DETAILED DESCRIPTION

The present disclosure relates to methods and compositions for treating viral infections (e.g., infections by a virus of the family coronaviridae, arenaviridae, and/or filoviridae) in a subject (e.g., a subject diagnosed with a viral infection or a subject at risk of infection by the virus). The disclosure is based, at least in part, upon the discovery that potent antiviral activity (e.g., viral inhibition) is achieved by administering an effective amount of a particular combination of compounds which synergistically inhibits the virus.

In one embodiment, the combination comprises (a) a nucleoside analogue acting as an inhibitor of the virally-encoded polymerase, e.g., remdesivir, an adenosine nucleoside triphosphate analog, (b) a drug efflux inhibitor (e.g., which prevents cytoplasmatic clearance of the nucleoside analogue by way of the ABC-family drug efflux transport system, e.g., a dual-specificity ABCB1/ABCG2 inhibitor, such as elacridar, tariquidar, zosuquidar (as well as other functionally equivalent ABCB1/ABCG2 dual inhibitors); and (c) an effective inducer of Heme Oxygenase 1 (the inducible form of heme oxygenases), such as an NRF2 agonist, e.g., curcumin, to lower the viral replication in the cell by means of removal of cytosolic heme. As demonstrated herein, administration of a combination of compounds representing the above-described three axes (i.e., hereinafter referred to as a “REC combination” or “REC compound”) achieves potent antiviral therapy against RNA-type viral pathogens.

Accordingly, the present disclosure provides antiviral therapies using the combined effects of agents which inhibit one or more of the above-described three aspects of viral replication. The present disclosure also provides beneficial routes of administration (e.g., oral and intravenous, and subcutaneous), as well as preferred administration regimens (e.g., administered simultaneously or sequentially). These and other embodiments are described below.

### Definitions

In order that the present invention may be more readily understood, certain terms are first defined. Additional definitions are set forth throughout the detailed description.

When trade names are used herein, such trade names independently include the trade name product and the active pharmaceutical ingredient(s) of the trade name product.

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

As used herein, the term “EC₅₀” refers to the concentration of a compound, or combination of compounds, which induces a response, either in an in vitro or an in vivo assay, which is 50% of the maximal response, i.e., halfway between the maximal response and the baseline.

As used herein, the term “effective amount” of an agent is that amount sufficient to effect beneficial or desired results, for example, clinical results, and, as such, an “effective amount” depends upon the context in which it is being applied. For example, in the context of administering an agent that inhibits a viral infection, an effective amount of an agent is, for example, an amount sufficient to achieve treatment or delay progression of the infection, as compared to the response obtained without administration of the agent. In some embodiments, a therapeutically effective amount is an amount of an agent to be delivered that is sufficient, when administered to a subject with a viral infection, to treat, improve symptoms of, prevent, and/or delay progression of the infection and/or condition.

As used herein, the terms “inhibits,” “blocks,” or “reduces” are used interchangeably and encompass both partial and complete inhibition/blocking. For example, the inhibition/blocking of a virus reduces or eliminates viral cell growth. As used herein, “inhibition”, “blocking”, or “reduces” are also intended to include any measurable decrease in biological function and/or activity of the virus, for example, when a combination or composition of the present invention is in contact with the virus, as compared to the virus not in contact with a combination or composition of the present invention. In some embodiments, a combination or composition inhibits or reduces viral growth and/or activity in a given system by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In other embodiments, a combination or composition inhibits or reduces viral growth and/or activity in a given system by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%.

As used herein, the term “inhibits growth” (e.g., referring to cells) is intended to include any measurable decrease in the growth of a cell, e.g., the inhibition of growth of a viral cell by at least about 10%, 20%, 30%, 40%, 50%, 60%, 70%, 80%, 90%, 99%, or 100%.

As used herein, a subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment (such as treatment with a combination or composition of the invention).

The term “in vivo” refers to processes that occur in a living organism.

As used herein, the term “patient” includes human and other mammalian subjects that receive either prophylactic or therapeutic treatment.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, a “pharmaceutically acceptable carrier” refers to, and includes, any and all solvents, dispersion media, coatings, antibacterial and antifungal agents, isotonic and absorption delaying agents, and the like that are physiologically compatible. The compositions can include a pharmaceutically acceptable salt, e.g., an acid addition salt or a base addition salt (see, e.g., Berge et al. (1977) J Pharm Sci 66:1-19).

As used herein, the term “preventing” when used in relation to a condition, refers to administration of a composition which reduces the frequency of, or delays the onset of, symptoms of a medical condition in a subject relative to a subject which does not receive the composition.

As used herein, the term “subject” includes any human or non-human animal. For example, the methods and combinations and compositions of the present invention can be used to treat a subject with a viral infection. The term “non-human animal” includes all vertebrates, e.g., mammals and non-mammals, such as non-human primates, sheep, dog, cow, chickens, amphibians, reptiles, etc.

As used herein, the terms “synergy” and “synergistic” refer to the effect achieved when the active ingredients (e.g., compounds) used together is greater than the sum of the effects that results from using the compounds separately. In some aspects, synergy results when a treatment outcome of the active ingredients used together is enhanced, augmented or improved over a treatment outcome of either compound individually. For example, in some aspects, synergy results in vivo when the effect of the active ingredients administered together (e.g., to a subject in need thereof as disclosed herein) provides an enhanced, augmented or improved treatment outcome in the subject, as compared to a treatment outcome when either compound is administered individually. For example, in some aspects synergy results in vivo when the effect of the active ingredients administered together inhibits, reduces or delays viral cell growth or extends or prolongs survival of a subject to a greater extent than the effect of either compound individually on viral cell growth or survival. For example, in some aspects, synergy results in vivo when a combined treatment with the active ingredients inhibits, reduces, or delays viral cell growth to a greater extent than the effect of either compound individually on viral cell growth. For example, in some aspects synergy results in vivo when a combined treatment with the active ingredients inhibits, reduces, or delays viral cell growth by at least 10%, at least 20%, at least 30%, at least 40%, at least 50%, at least 60%, at least 70%, at least 80%, at least 90%, or at least 100% or more than the effect of either compound individually on viral cell growth. In some aspects, synergy results in vivo when a combined treatment with the active ingredients extends or prolongs survival in a subject to a greater extent than the survival which results from treatment with either compound individually. For example, in some aspects, synergy results in vivo when a combined treatment with the active ingredients extends, or prolongs the survival in a subject by at least 5 days, at least 10 days, at least 20 days, at least 30 days, at least 40 days, at least 50 days or more than the survival which results from treatment with either compound individually.

In some embodiments, a synergistic effect is attained when the active ingredients (e.g., compounds) are: (1) co-formulated and administered or delivered simultaneously in a combined, unit dosage formulation; (2) delivered by alternation or in parallel as separate formulations; or (3) by some other regimen. When delivered in alternation therapy, a synergistic effect is attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

As used herein, the terms “therapeutically effective amount” or “therapeutically effective dose,” or similar terms used herein are intended to mean an amount of an agent (e.g., a combination of compounds of composition that inhibit viral infections) that will elicit the desired biological or medical response (e.g., viral cell death).

The terms “treat,” “treating,” and “treatment,” as used herein, refer to therapeutic or preventative measures described herein. The methods of “treatment” employ administration to a subject, in need of such treatment, a combination or composition of the present disclosure, for example, a subject diagnosed with a viral infection or a subject who is at risk of a viral infection, in order to prevent, cure, delay, reduce the severity of, or ameliorate one or more symptoms of the infection, or in order to prolong the survival of a subject beyond that expected in the absence of such treatment.

Viruses

Viruses can infect all types of life forms, from animals and plants to microorganisms, including bacteria and archaea. More than 6,000 virus species have been described in detail. When infected, a host cell is forced to rapidly produce thousands of identical copies of the original virus. When not inside an infected cell or in the process of infecting a cell, viruses exist in the form of independent particles, or virions, consisting of: (i) the genetic material, i.e. long molecules of DNA or RNA that encode the structure of the proteins by which the virus acts; (ii) a protein coat, the capsid, which surrounds and protects the genetic material; and in some cases (iii) an outside envelope of lipids. The shapes of these virus particles range from simple helical and icosahedral forms to more complex structures. Most virus species have virions too small to be seen with an optical microscope as they are one hundredth the size of most bacteria.

According to the ICTV classification system (in conjunction with the Baltimore classification system), classification of viruses is based on the mechanism of mRNA production. Viruses must generate mRNAs from their genomes to produce proteins and replicate themselves, but different mechanisms are used to achieve this in each virus family. Viral genomes may be single-stranded (ss) or double-stranded (ds), RNA or DNA, and may or may not use reverse transcriptase (RT). In addition, ssRNA viruses may be either sense (+) or antisense (−). This classification places viruses into the following seven groups:

TABLE 1
I: dsDNA viruses (e.g. Adenoviruses, Herpesviruses, Poxviruses)
II: ssDNA viruses (+ strand or “sense”) DNA (e.g. Parvoviruses)
III: dsRNA viruses (e.g. Reoviruses)
IV: (+)ssRNA viruses (+ strand or sense) RNA (e.g. Coronaviruses, Picornaviruses,
Togaviruses)
V: (−)ssRNA viruses (− strand or antisense) RNA (e.g. Orthomyxoviruses,
Rhabdoviruses)
VI: ssRNA-RT viruses (+ strand or sense) RNA with DNA intermediate in life-cycle (e.g.
Retroviruses)
VII: dsDNA-RT viruses DNA with RNA intermediate in life-cycle (e.g. Hepadnaviruses)

Coronaviruses are a group of related RNA viruses that cause diseases in mammals and birds. In humans, these viruses cause respiratory tract infections that can range from mild to lethal. Mild illnesses include some cases of the common cold (which is also caused by other viruses, predominantly rhinoviruses), while more lethal varieties can cause SARS, MERS, and COVID-19. Symptoms in other species vary: in chickens, they cause an upper respiratory tract disease, while in cows and pigs they cause diarrhea. There are as yet no vaccines or antiviral drugs to prevent or treat human coronavirus infections.

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

The scientific name for coronavirus is Orthocoronavirinae or Coronavirinae. Coronaviruses belong to the family of Coronaviridae, order Nidovirales, and realm Riboviria. They are divided into alphacoronaviruses and betacoronaviruses which infect mammals—and gammacoronaviruses and deltacoronaviruses, which primarily infect birds. A classification of coronavinis is shown in Table 2.

TABLE 2
Genus: Alphacoronavirus; type species: Alphacoronavirus 1 (TGEV)
Species: Alphacoronavirus 1, Human coronavirus 229E, Human coronavirus
NL63, Miniopterus bat coronavirus 1, Miniopterus bat coronavirus HKU8,
Porcine epidemic diarrhea virus, Rhinolophus bat coronavirus HKU2, Scotophilus
bat coronavirus 512
Genus Betacoronavirus; type species: Murine coronavirus (MHV)
Species: Betacoronavirus 1 (Bovine Coronavirus, Human coronavirus OC43),
Hedgehog coronavirus 1, Human coronavirus HKU1, Middle East respiratory
syndrome-related coronavirus, Murine coronavirus, Pipistrellus bat coronavirus
HKU5, Rousettus bat coronavirus HKU9, Severe acute respiratory syndrome-
related coronavirus (SARS-COV, SARS-COV-2), Tylonycteris bat coronavirus
HKU4
Genus Gammacoronavirus; type species: Avian coronavirus (IBV)
Species: Avian coronavirus, Beluga whale coronavirus SW1
Genus Deltacoronavirus; type species: Bulbul coronavirus HKU11
Species: Bulbul coronavirus HKU11, Porcine coronavirus HKU15

An arenavirus is a bisegmented ambisense RNA virus that is a member of the family Arenaviridae. Arenaviruses have a segmented RNA genome that consists of two single-stranded ambisense RNAs. As with all negative-sense RNA viruses, the genomic RNA alone is not infectious and the viral replication machinery is required to initiate infection within a host cell. Genomic sense RNA packaged into the arenavirus virion is designated negative-sense RNA, and must first be copied into a positive-sense mRNA in order to produce viral protein. The two RNA segments are denoted Small (S) and Large (L), and code for four viral proteins in a unique ambisense coding strategy. Each RNA segment codes for two viral proteins in opposite orientation such that the negative-sense RNA genome serves as the template for transcription of a single mRNA and the positive-sense copy of the RNA genome templates a second mRNA. The separate coding sequences of the two viral proteins are divided by an intergenic region RNA sequence that is predicted to fold into a stable hairpin structure.

These viruses infect rodents and occasionally humans. A class of novel, highly divergent arenaviruses, properly known as reptarenaviruses, have also been discovered which infect snakes to produce inclusion body disease. At least eight arenaviruses are known to cause human disease. The diseases derived from arenaviruses range in severity. Aseptic meningitis, a severe human disease that causes inflammation covering the brain and spinal cord, can arise from the lymphocytic choriomeningitis virus. Hemorrhagic fever syndromes, including Lassa fever, are derived from infections such as Guanarito virus, Junin virus, Lassa virus, Lujo virus,[2] Machupo virus, Sabia virus, or Whitewater Arroyo virus. Because of the epidemiological association with rodents, some arenaviruses and bunyaviruses are designated as roboviruses.

Within the family Arenaviridae, arenaviruses were formerly all placed in the genus Arenavirus, but in 2014 were divided into the genera Mammarenavirus for those with mammalian hosts and Reptarenavirus for those infecting snakes. Reptarenaviruses and mammarenavirus are separated by an impenetrable species barrier. Infected rodents cannot pass disease onto snakes, and IBD in captive snakes is not transmissible to humans. A third genus, Hartmanivirus, has also been established, including other species that infect snakes. The organisation of the genome of this genus is typical of arenaviruses but their glycoproteins resemble those of filoviruses. Species in this genus lack the matrix protein. A fourth genus, Antennavirus has also been established to accommodate two arenaviruses found in striated frogfish (Antennarius striatus).

Mammarenaviruses can be divided into two serogroups, which differ genetically and by geographical distribution: When the virus is classified “Old World” this means it was found in the Eastern Hemisphere in places such as Europe, Asia, and Africa. When it is found in the Western Hemisphere, in places such as Argentina, Bolivia, Venezuela, Brazil, and the United States, it is classified “New World”. Lymphocytic choriomeningitis (LCM) virus is the only arenavirus to exist in both areas but is classified as an Old World virus.

The family Filoviridae, a member of the order Mononegavirales, is the taxonomic home of several related viruses (filoviruses or filovirids) that form filamentous infectious viral particles (virions) and encode their genome in the form of single-stranded negative-sense RNA. Two members of the family that are commonly known are Ebola virus and Marburg virus. Both viruses, and some of their lesser known relatives, cause severe disease in humans and nonhuman primates in the form of viral hemorrhagic fevers. Table 3 provides the genus, species, and virus names for the family Filoviridae.

TABLE 3
Family Filoviridae

| Genus name | Species name | Virus name (abbreviation) |
|---|---|---|
| Cuevavirus | Lloviu cuevavirus | Lloviu virus (LLOV) |
| Dianlovirus | | M\check{e}nglà virus (MLAV) |
| Ebolavirus | Bombali ebolavirus | Bombali virus |
| | Bundibugyo ebolavirus | Bundibugyo virus (BDBV; |
| | | previously BEBOV) |
| | Reston ebolavirus | Reston virus (RESTV; |
| | | previously REBOV) |
| | Sudan ebolavirus | Sudan virus (SUDV; |
| | | previously SEBOV) |
| | Taï Forest ebolavirus | Taï Forest virus (TAFV; |
| | | previously CIEBOV) |
| | Zaire ebolavirus | Ebola virus (EBOV; |
| | | previously ZEBOV) |
| Marburgvirus | Marburg marburgvirus | Marburg virus (MARV) |
| | | Ravn virus (RAVV) |

Viral Polymerase Inhibitors

Viral polymerases play a central role in viral genome replication and transcription. Based on the genome type and the specific needs of particular virus, RNA-dependent RNA polymerase, RNA-dependent DNA polymerase, DNA-dependent RNA polymerase, and DNA-dependent RNA polymerases are found in various viruses. Viral polymerases are generally active as a single protein capable of carrying out multiple functions related to viral genome synthesis. Specifically, viral polymerases use variety of mechanisms to recognize initial binding sites, ensure processive elongation, terminate replication at the end of the genome, and also coordinate the chemical steps of nucleic acid synthesis with other enzymatic activities.

The present invention provides methods for treating a subject infected with a virus by administering a combination of compounds which comprises a viral polymerase inhibitor, as well as compositions, e.g., a pharmaceutical composition, which comprise a viral polymerase inhibitor. Examples of such inhibitors include remdesivir (Veklury®), and other functionally similar nucleoside analogs, i.e., nucleoside analogs capable of interfering with the action of viral RNA-dependent RNA polymerase and evading proofreading by viral exoribonuclease (ExoN), thus causing a decrease in viral RNA production (Li et al., Drug Discov. Ther. (2020); 14(2):73; Ferner and Aronson, BMJ(2020) 369:m1610). For example, remdesivir, a compound of Formula II

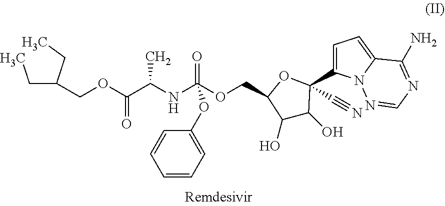

diffuses into cells where it is converted to GS-441524 mono-phosphate via the actions of esterases (CES1 and CTSA) and a phosphoamidase (HINT1); this in turn is further phosphorylated to its active metabolite triphosphate by nucleoside-phosphate kinases. Further information regarding remdesivir and its functionally similar analogs is known in the art, e.g., US 20190255085, U.S. Pat. Nos. 10,251,904, 9,724,360, US 20160122374, and WO 2017049060, which are incorporated herein by reference.

As demonstrated in the present invention, the specificity of remdesivir in the REC combination is remarkable. Various other ribonucleoside analogues (EIDD1931, EIDD2801, Galidesivir, Favipiravir, Rimonavir) were tested, but none were able to substitute for remdesivir. The functional metabolite of remdesivir, GS441524, also failed to substitute for remdesivir. Accordingly, the cellular uptake of remdesivir is more effective than GS441524 and only the pro-drug is subjected to ABC-family export. Considering that remdesivir is currently administered clinically through i.v. infusion mainly due to first-pass hepatic clearing, the enhanced potency of remdesivir attainable by use of ABC-dual inhibition advantageously makes it capable of being administered orally which, in turn, allows it to attain greater potency, and thus extend the use of the drug.

In addition to the data provided herein regarding the REC combination, additional empirical evidence is shown for multiple drugs currently being explored for efficacy against SARS-CoV2. The initial HD-DoE screen included two focus compounds—Favipiravir, and Hydroxychloroquine. Both drugs failed to emerge as efficacious, individually, and in combination with others, against SARS-CoV2 in the VERO6 EGFP assay. Irbesartan, Camostat, Ritonavir, Lopinavir, Rimantadine, EIDD1931, Mefloquine, Arbidol, are all candidate drugs against SARS-CoV-2 and COVID-19. (McKee et al. (2020); Pharmacol. Res.), however, this study did not provide evidence for efficacy. The antihelminth drug Ivermectin, which is currently undergoing clinical testing in COVID-19 (e.g. NCT04351347, NCT04392713, NCT04360356), displayed individual, but limited potency against SARS-CoV2, but was unable to enhance the REC combination.

Efflux Inhibitors

Most microorganisms have highly conserved DNA sequences in their genome that are transcribed and translated to efflux pumps. Efflux pumps are capable of moving a variety of different toxic compounds out of cells, such as antibiotics, heavy metals, organic pollutants, plant-produced compounds, quorum sensing signals, bacterial metabolites and neurotransmitters via active efflux, which is vital part for xenobiotic metabolism. This active efflux mechanism is responsible for various types of resistance to bacterial pathogens within bacterial species—the most concerning being antibiotic resistance because microorganisms can have adapted efflux pumps to divert toxins out of the cytoplasm and into extracellular media. Multidrug efflux pumps represent one of the major mechanisms of drug resistance in bacteria

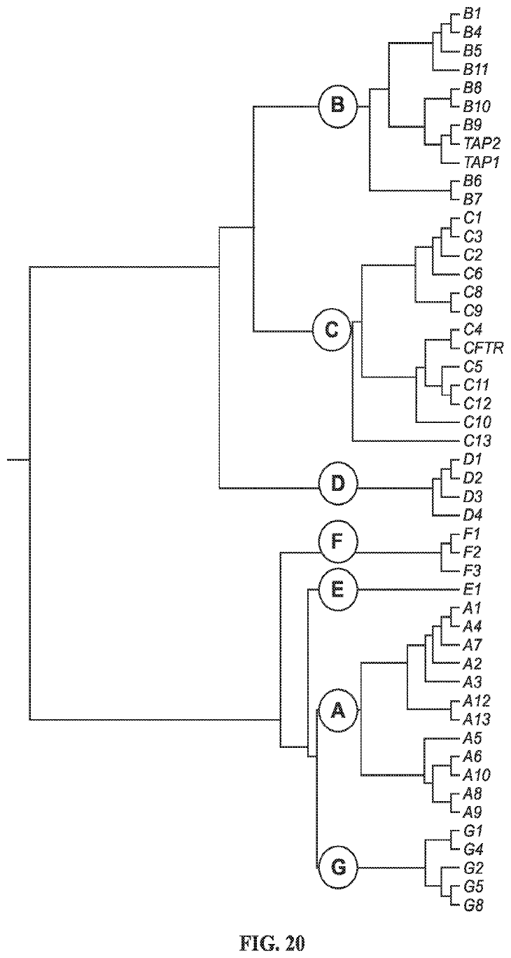

Mammalian cells also have highly conserved DNA sequences in their genome that are transcribed and translated into efflux pumps and these proteins are evolutionary descendants of the prokaryotic transporters and are thus ancient proteins. All of the efflux transporters belong to the family of ATP-powered pumps. ATP-binding cassette (ABC) transporters are an example of ATP-dependent pumps. ABC transporters are ubiquitous membrane-bound proteins, present in all prokaryotes, as well as plants, fungi, yeast and animals. These pumps can move substrates in (influx) or out (efflux) of cells. ABC-family transporters are responsible for molecular efflux of multiple drug classes. An overview of the mammalian ABC-family is provided in Hum Genomics. 2009 April; 3(3):281-90. doi: 10.1186/1479-7364-3-3-281. The human genome contains 49 ABC genes, arranged in eight subfamilies and named via divergent evolution (as described in ncbi.nlm.nih.gov/pmc/articles/instance/3523235/bin/1479-7364-3-3-281-1.jpg which is shown in FIG. 20). Human ABC gene subfamilies are listed in Table 4.

TABLE 4
Human ABC gene subfamilies

| Subfamily name | Aliases Number of genes | Number of pseudogenes |
|---|---|---|
| ABCA ABC1 | 12 | 5 |
| ABCB MDR | 11 | 4 |
| ABCC MRP | 13 | 2 |
| ABCD ALD | 4 | 4 |
| ABCE OABP | 1 | 2 |
| ABCF GGN20 | 3 | 2 |
| ABCG White | 5 | 2 |
| Total | 49 | 21 |

The combination therapy of the present invention includes administration of at least inhibitor of Subfamily B of the ABC family (ABCB) of efflux transporters, preferably dual inhibitors of Subfamily B and Subfamily G (i.e., an inhibitor with dual specificity). This subfamily of 11 genes is unique to mammals and includes four full-transporters and seven half-transporters. Several of the B family members are known to confer multidrug resistance in cancer cells. Hence, subfamily B has also been called the “MDR family of ABC transporters.” The MDR name reflects the fact that ABCB-family efflux transporters are involved in export of known pharmaceutical compounds, which to the body is deemed foreign, and thus exported. In certain cases, the export through the ABC-family leads to an effective lowering of the intracellular concentration of the pharmaceutical agent, such as chemotherapeutic substances (nucleoside analogs), which would be exported by the cancer cells, effectively lowering their potency. Similarly, according to the present invention, the ABC-inhibitory drug (e.g., elacridar) increases the potency of remdesivir.

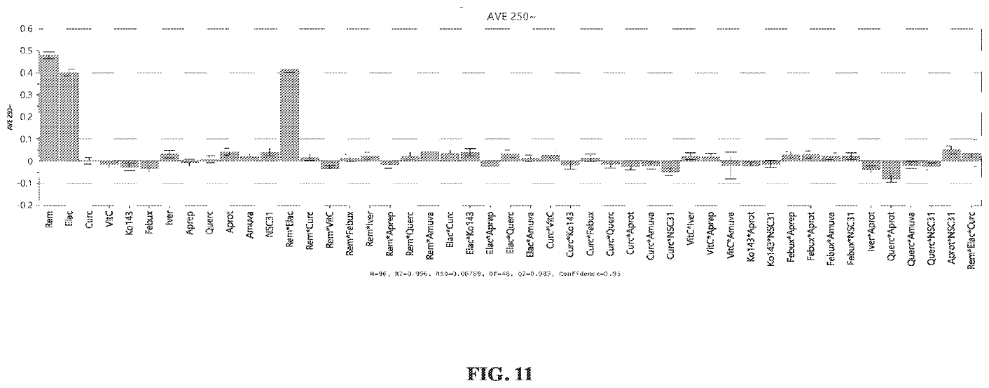

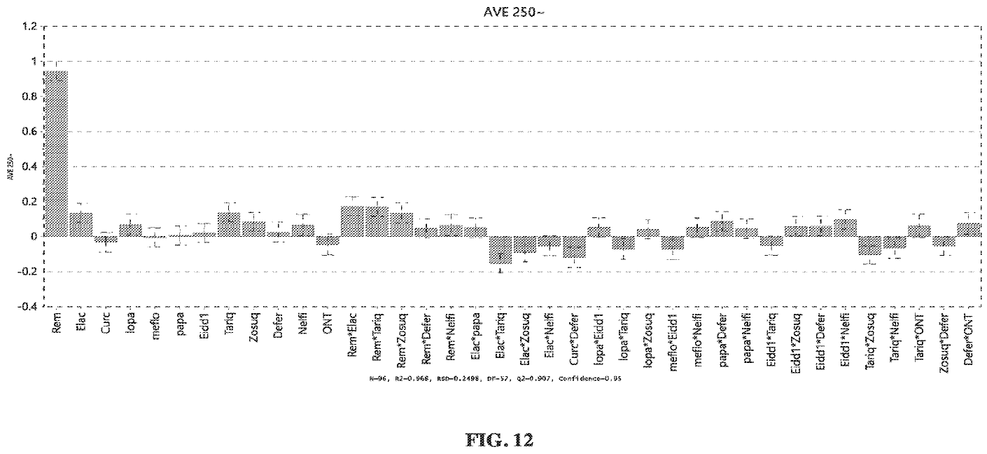

Elacridar is a third-generation P-glycoprotein non-competitor inhibitor (ABCB1) which also inhibits the ABCG2 protein. As demonstrated herein, the specificity of elacridar to prevent remdesivir export was determined through additional testing. Inhibition of ABCG2 using the ABCG2-specific inhibitor Ko143 failed to complement for elacridar (FIG. 11). Furthermore, Febuxostat, another inhibitor of ABCG2, failed to complement (FIG. 11). However, tariquidar, a low-nM dual-specificity ABCB1 (5.1 nM)/ABCG2 inhibitor substituted for elacridar (FIG. 12), as did zosuquidar. Equivalent interaction terms were observed to remdesivir (Rem*Elac, Rem*Tariq, Rem*Zosuq), (FIG. 12). Quercetin is a known ABCB1 inhibitor but operates at a higher concentration than elacridar/tariquidar/zosuquidar. It failed to complement for elacridar (FIG. 11). Similarly, ONT-093 is specific for ABCB1 over ABCG2, and failed to complement (FIG. 12). Vardenafil was also found to be a potent inhibitor of ABCB1 (Ding et al. (2011) The phosphodiesterase-5 inhibitor vardenafil is a potent inhibitor of ABCB1/P-glycoprotein transporter. PLoS One) and also failed to complement elacridar (data not shown). Accordingly, to effectively abrogate remdesivir drug efflux dual inhibitors of ABCG2/ABCB1 are particularly preferred for use in the present invention.

Accordingly, the present invention provides methods for treating a subject infected with a virus by administering a combination of compounds which comprises remdesivir in combination with one or more efflux inhibitors, preferably a dual-specificity ABCB1/ABCG2 channel transport inhibitor, which reduces or prevents cytoplasmatic clearance of remdesivir by way of the ABC-family drug efflux transport system (i.e., ABCB1/ABCG2). In one embodiment, the efflux inhibitor is elacridar which is a P-Glycoprotein/ABC-family channel transport inhibitor, i.e., a compound of Formula I;

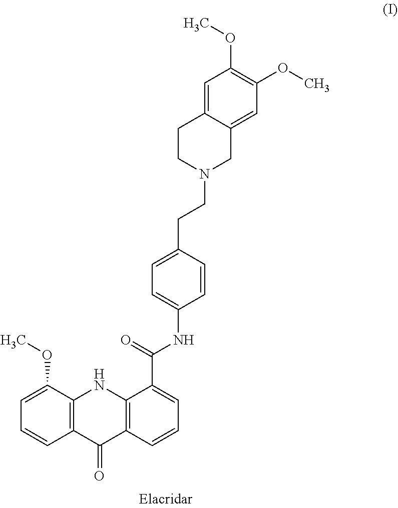

Accordingly, other members of this class of inhibitors also can be used in the invention. This includes, e.g., specific inhibitors against the ABCB1 drug efflux transporter, in particular, inhibitors with dual specificity against ABCB1 and ABCG2, such as tariquidar or zosuquidar, or P-Glycoprotein Inhibitor, C-4 (CTK8G2344), or HM30181AK. The P-glycoprotein family is known as drug-efflux transporters and serve to eliminate foreign drugs in a cell by an energy dependent (ATP-consuming) transport. The class of proteins is encoded by ABC-family genes, and consists of multiple members, displaying distinct specificities against compounds.

Other examples of such inhibitors include the ABCB1 inhibitor class (P-glycoprotein inhibitors), such as tariquidar, zosuquidar, and other functionally equivalent inhibitors as shown in Table 5. Not all of the listed compounds operate at low nM efficiency and many of the listed compounds are substrates for the ABCB1 transporter and operate as independent pharmaceutical agents on their own. Consequently, for many of the drugs capable of interfering with ABCB1 function, such may be undesirable in the REC combination. Accordingly, a preferred inhibitor is an ABCB1/ABCG2 inhibitor having limited (or no) pharmaceutical effects on the organism, and thus displaying a minimal pharmacodynamic profile, yet able to elicit high ABCB1/ABCG2 inhibition at low concentration, thus displaying a low and sustained pharmacokinetic profile attained by low frequency administration (such as daily and oral administration).

TABLE 5

| DRUG | DRUG DESCRIPTION |
|---|---|
| Verapamil | A non-dihydropyridine calcium channel blocker used in the treatment of |
| | angina, arrhythmia, and hypertension. |
| Saquinavir | An HIV protease inhibitor used in combination with other antiretroviral |
| | agents for the treatment of HIV-1 with advanced immunodeficiency. |
| Indinavir | A protease inhibitor used to treat HIV infection. |
| Reserpine | For the treatment of hypertension |
| Nifedipine | A dihydropyridine calcium channel blocker indicated for the management |
| | of several subtypes of angina pectoris, and hypertension. |
| Mifepristone | A cortisol receptor blocker used to treat Cushing's syndrome, and to |
| | terminate pregnancies up to 70 days gestation. |
| Dexamethasone | A glucocorticoid available in various modes of administration that |
| | is used for the treatment of various inflammatory conditions, including bronchial asthma, as well |
| | as endocrine and rheumatic disorders. |
| Clotrimazole | A topical broad-spectrum antifungal agent used for the treatment of a wide |
| | variety of dermatophyte infections and candidiasis. |
| Ritonavir | An HIV protease inhibitor used in combination with other antivirals in the |
| | treatment of HIV infection. |
| Digoxin | A cardiac glycoside used in the treatment of mild to moderate heart failure |
| | and for ventricular response rate control in chronic atrial fibrillation. |
| Trimethoprim | An antifolate antibiotic often used in combination with sulfamethoxazole |
| | to treat a number of infections, including those of the urinary tract, respiratory tract, and |
| | gastrointestinal tract. |
| Progesterone | A hormone used for a variety of functions, including contraception, |
| | control of abnormal uterine bleeding, maintenance of pregnancy, and prevention of endometrial |
| | hyperplasia. |
| Tacrolimus | A calcineurin inhibitor used to prevent organ transplant rejection and to |
| | treat moderate to severe atopic dermatitis. |
| Erythromycin | A macrolide antibiotic used to treat and prevent a variety of bacterial |
| | infections. |
| Amiodarone | A class III antiarrhythmic indicated for the treatment of recurrent |
| | hemodynamically unstable ventricular tachycardia and recurrent ventricular fibrillation. |
| Tamoxifen | A selective estrogen receptor modulator used to treat estrogen receptor |
| | positive breast cancer, reduce the risk of invasive breast cancer following surgery, or reduce the |
| | risk of breast cancer in high risk women. |
| Mitoxantrone | A chemotherapeutic agent used for the treatment of secondary progressive, |
| | progressive relapsing, or worsening relapsing-remitting multiple sclerosis. |
| Daunorubicin | An anthracycline aminoglycoside used to induce remission of |
| | nonlymphocytic leukemia and acute lymphocytic leukemia. |
| Quinidine | A medication used to restore normal sinus rhythm, treat atrial fibrillation |
| | and flutter, and treat ventricular arrhythmias. |
| Ketoconazole | A broad spectrum antifungal used to treat seborrheic dermatitis and fungal |
| | skin infections. |
| Mibefradil | For the treatment of angina and high blood pressure. |
| Cholesterol | The principal sterol of all higher animals, distributed in body tissues, |
| | especially the brain and spinal cord, and in animal fats and oils. |
| Mefloquine | An antimalarial agent used as a schizonticidal agent. |
| Nicardipine | A calcium channel blocker used for the short-term treatment of |
| | hypertension and chronic stable angina. |
| Amlodipine | A calcium channel blocker used to treat hypertension and angina. |
| Testosterone | A hormone used to treat hypogonadism, breast carcinoma in women, or |
| | the vasomotor symptoms of menopause. |
| Azelastine | A histamine H1-receptor antagonist used intranasally to treat allergic and |
| | vasomotor rhinitis and in an ophthalmic solution to treat allergic conjunctivitis. |
| Ranitidine | A histamine H2 antagonist used to treat duodenal ulcers, Zollinger-Ellison |
| | syndrome, gastric ulcers, GERD, and erosive esophagitis. |
| Etoposide | A podophyllotoxin derivative used to treat testicular and small cell lung |
| | tumors. |
| Methadone | An opioid analgesic indicated for management of severe pain that is not |
| | responsive to alternative treatments. Also used to aid in detoxification and maintenance |
| | treatment of opioid addiction. |
| Simvastatin | An HMG-CoA reductase inhibitor used to lower lipid levels and reduce |
| | the risk of cardiovascular events including myocardial infarction and stroke. |
| Lovastatin | An HMG-CoA reductase inhibitor used to lower LDL cholesterol and |
| | reduce the risk of cardiovascular disease and associated conditions, |
| | including myocardial infarction and stroke. |
| Atorvastatin | An HMG-COA reductase inhibitor used to lower lipid levels and reduce |
| | the risk of cardiovascular disease including myocardial infarction and stroke. |
| Paclitaxel | A taxoid chemotherapeutic agent used as first-line and subsequent therapy |
| | for the treatment of advanced carcinoma of the ovary, and other various cancers including breast |
| | and lung cancer. |
| Prazosin | An alpha-blocker that causes a decrease in total peripheral resistance and |
| | is used to treat hypertension. |
| Nitrendipine | For the treatment of mild to moderate hypertension |
| Dipyridamole | A phosphodiesterase inhibitor used to prevent postoperative |
| | thromboembolic events. |
| Diltiazem | A calcium channel blocker used to treat hypertension and to manage |
| | chronic stable angina. |
| Clarithromycin | A macrolide antibiotic used for the treatment of a wide variety of |
| | bacterial infections such as acute otitis, pharyngitis, tonsillitis, respiratory tract infections, |
| | uncomplicated skin infections, and helicobacter pylori infection. |
| Chlorpromazine | A phenothiazine antipsychotic used to treat nausea, vomiting, |
| | preoperative anxiety, schizophrenia, bipolar disorder, and severe behavioral problems in |
| | children. |
| Dactinomycin | An actinomycin used to treat a wide variety of cancers. |
| Troleandomycin | For the treatment of bacterial infection. |
| Trifluoperazine | A phenothiazine used to treat depression, anxiety, and agitation. |
| Terfenadine | For the treatment of allergic rhinitis, hay fever, and allergic skin disorders. |
| Quinine | An alkaloid used to treat uncomplicated Plasmodium falciparum malaria. |
| Itraconazole | An antifungal agent used for the treatment of various fungal infections in |
| | immunocompromised and non-immunocompromised patients, such as pulmonary and |
| | extrapulmonary blastomycosis, histoplasmosis, and onychomycosis. |
| Indomethacin | A nonsteroidal anti-inflammatory (NSAID) used for symptomatic |
| | management of chronic musculoskeletal pain conditions and to induce closure of a |
| | hemodynamically significant patent ductus arteriosus in premature infants. |
| Colforsin | Potent activator of the adenylate cyclase system and the biosynthesis of |
| | cyclic AMP. From the plant Coleus forskohlii. Has antihypertensive, |
| | positive inotropic, platelet aggregation inhibitory, and smooth muscle relaxant activities; . . . |
| Fluphenazine | A phenothiazine used to treat patients requiring long-term neuroleptic |
| | therapy. |
| Felodipine | A calcium channel blocker used to treat hypertension. |
| Clofazimine | A riminophenazine antimycobacterial used to treat leprosy. |
| Carvedilol | A non selective beta-adrenergic antagonist used to treat mild to severe |
| | chronic heart failure, hypertension, and left ventricular dysfunction following myocardial |
| | infarction in clinically stable patients. |
| Lidocaine | A local anesthetic used in a wide variety of superficial and invasive |
| | procedures. |
| Azithromycin | A macrolide antibiotic used to treat a variety of bacterial infections. |
| Acetaminophen | An analgesic drug used alone or in combination with opioids for |
| | pain management, and as an antipyretic agent. |
| Fluconazole | A triazole antifungal used to treat various fungal infections including |
| | candidiasis. |
| Pantoprazole | A proton pump inhibitor used to treat erosive esophagitis, gastric acid |
| | hypersecretion, and to promote healing of tissue damage caused by gastric acid. |
| Omeprazole | A proton pump inhibitor used to treat GERD associated conditions such as |
| | heartburn and gastric acid hypersecretion, and to promote healing of tissue damage and ulcers |
| | caused by gastric acid and H. pylori infection. |
| Lansoprazole | A proton pump inhibitor used to help gastrointestinal ulcers heal, to treat |
| | symptoms of gastroesophageal reflux disease (GERD), to eradicate Helicobacter pylori, and to |
| | treat hypersecretory conditions such as Zollinger-Ellison Syndrome. |
| Losartan | An angiotensin receptor blocker used to treat hypertension and diabetic |
| | nephropathy, and is used to reduce the risk of stroke. |
| Enalapril | A prodrug of an ACE inhibitor used to treat hypertension and congestive |
| | heart failure. |
| Doxazosin | An alpha-1 adrenergic receptor used to treat mild to moderate |
| | hypertension and urinary obstruction due to benign prostatic hyperplasia. |
| Cilazapril | An ACE inhibitor used for the management of hypertension and heart |
| | failure. |
| Captopril | An ACE inhibitor used for the management of essential or renovascular |
| | hypertension, congestive heart failure, left ventricular dysfunction following myocardial |
| | infarction, and nephropathy. |
| Candesartan cilexetil | An angiotensin receptor blocker used to treat hypertension, systolic |
| | hypertension, left ventricular hypertrophy, and delay progression of diabetic nephropathy. |
| Miconazole | An azole antifungal with broad-spectrum activity used to treat fungal |
| | infections affecting the vagina, mouth and skin, including candidiasis. |
| Ergotamine | A alpha-1 selective adrenergic agonist vasoconstrictor used to treat |
| | migraines with or without aura and cluster headaches. |
| Ergometrine | An ergot alkaloid used for the treatment of postpartum haemorrhage and |
| | post abortion hemorrhage in patients with uterine atony. |
| Dihydroergotamine | An ergot alkaloid used in the acute treatment of migraine headache |
| | and cluster headache. |
| Bromocriptine | A dopamine D2 receptor agonist used for the treatment of galactorrhea |
| | due to hyperprolactinemia and other prolactin-related conditions, as well as in early Parkinsonian |
| | Syndrome. |
| Fentanyl | An opioid analgesic used in anesthesia, for breakthrough cancer pain, or |
| | round the clock pain management. |
| Alfentanil | An opioid agonist used to induce and maintain anesthesia, as well as an |
| | analgesic. |
| Nisoldipine | A calcium channel blocker used as monotherapy or combined with other |
| | drugs for the treatment of hypertension. |
| Haloperidol | An antipsychotic agent used to treat schizophrenia and other psychoses, as |
| | well as symptoms of agitation, irritability, and delirium. |
| Fluvoxamine | A selective serotonin-reuptake inhibitor used to treat obsessive- |
| | compulsive disorder. |
| Flurazepam | A long-acting benzodiazepine with a rapid onset of action that is |
| | commonly used to treat insomnia. |
| Fluoxetine | A selective serotonin reuptake inhibitor used to treat major depressive |
| | disorder, bulimia, OCD, premenstrual dysphoric disorder, panic disorder, and bipolar I. |
| Desipramine | A tricyclic antidepressant used in the treatment of depression. |
| Clomipramine | A tricyclic antidepressant used in the treatment of obsessive-compulsive |
| | disorder and disorders with an obsessive-compulsive component, such as depression, |
| | schizophrenia, and Tourette's disorder. |
| Chlorprothixene | For treatment of psychotic disorders (e.g. schizophrenia) and of |
| | acute mania occuring as part of bipolar disorders. |
| Buspirone | An anxiolytic agent used for short-term treatment of generalized anxiety |
| | and second-line treatment of depression. |
| Pimozide | An antipsychotic used to manage debilitating motor and phonic tics in |
| | patients with Tourette's Disorder. |
| Ivermectin | An anti parasite medication used to treat head lice, onchocerciasis, |
| | strongyloidiasis, ascariasis, trichuriasis, and enterobiasis. |
| Astemizole | A second generation antihistamine used to treat allergy symptoms. |
| Triflupromazine | Used mainly in the management of psychoses. Also used to control |
| | nausea and vomiting. |
| Protriptyline | A tricyclic antidepressant that is indicated in the treatment of depression |
| | only under close clinical supervision. |
| Bepridil | For the treatment of hypertension, and chronic stable angina (classic |
| | effort-associated angina). |
| Loratadine | A second generation antihistamine used to manage the symptoms of |
| | allergic rhinitis. |
| Desloratadine | A second generation tricyclic antihistamine used to treat seasonal and non |
| | seasonal allergic rhinitis, pruritus, and urticaria. |
| Sertraline | A selective serotonin reuptake inhibitor (SSRI) indicated to treat major |
| | depressive disorder, social anxiety disorder and many other psychiatric conditions. |
| Reboxetine | For the treatment of clinical depression. |
| Paroxetine | A selective serotonin reuptake inhibitor used to treat major depressive |
| | disorder, panic disorder, OCD, social phobia, generalized anxiety disorder, the vasomotor |
| | symptoms of menopause, and premenstrual dysphoric disorder. |
| Citalopram | A selective serotonin reuptake inhibitor (SSRI) used in the treatment of |
| | depression. |
| Estramustine | An antineoplastic agent used for the management of metastatic and/or |
| | progressive prostate cancer in palliative setting. |
| Flupentixol | A thioxanthene neuroleptic used to treat schizophrenia and depression. |
| Megestrol acetate | A progestin that is administered orally to treat anorexia and |
| | cachexia or serious unexplained weight loss and is also used as an antineoplastic agent to treat |
| | certain types of malignancy. |
| Staurosporine | An indolocarbazole that is a potent protein kinase C inhibitor which |
| | enhances cAMP-mediated responses in human neuroblastoma cells. (Biochem Biophys Res |
| | Commun 1995; 214(3): 1114-20) |
| Quinacrine | For the treatment of giardiasis and cutaneous leishmaniasis and the |
| | management of malignant effusions. |
| Genistein | Currently Genistein is being studied in clinical trials as a treatment for |
| | prostate cancer. |
| Quercetin | Quercetin is a flavonol widely distributed in plants. It is an antioxidant, |
| | like many other phenolic heterocyclic compounds. Glycosylated forms include RUTIN and |
| | quercetrin. |
| Levofloxacin | A fluoroquinolone antibiotic used to treat infections caused by susceptible |
| | bacteria of the upper respiratory tract, skin and skin structures, urinary tract, and prostate, as well |
| | as for post-exposure treatment of inhaled anthrax and the plague. |
| Glyburide | A sulfonylurea used in the treatment of non insulin dependent diabetes |
| | mellitus. |
| Amsacrine | A cytotoxic agent used to induce remission in acute adult leukemia that is |
| | not adequately responsive to other agents. |
| Gramicidin D | A bactericidal antibiotic used in the treatment of dermatological and |
| | ophthalmic infections. |
| Benzocaine | A topical local anesthetic used for the temporary relief of pain and itching |
| | associated with minor burns, sunburn, scrapes and insect bites or minor skin irritations. |
| Lopinavir | An HIV-1 protease inhibitor used in combination with ritonavir to treat |
| | human immunodeficiency virus (HIV) infection. |
| Caspofungin | An echinocandin used to treat a variety of fungal infections. |
| Imatinib | A tyrosine kinase inhibitor used to treat a number of leukemias, |
| | myelodysplastic/myeloproliferative disease, systemic mastocytosis, hypereosinophilic syndrome, |
| | dermatofibrosarcoma protuberans, and gastrointestinal stromal tumors. |
| Sorafenib | A kinase inhibitor used in the treatment of unresectable liver carcinoma |
| | and advanced renal carcinoma. |
| Sunitinib | A receptor tyrosine kinase inhibitor and chemotherapeutic agent used for |
| | the treatment of renal cell carcinoma (RCC) and imatinib-resistant gastrointestinal stromal tumor |
| | (GIST). |
| Dronabinol | A synthetic delta-9-THC used in the treatment of anorexia and weight loss |
| | in HIV patients as well as nausea and vomiting in cancer chemotherapy. |
| Buprenorphine | A partial opioid agonist used for management of severe pain that is not |
| | responsive to alternative treatments. Also used for maintenance treatment of opioid addiction. |
| Telmisartan | An ARB used to treat hypertension, diabetic nephropathy, and congestive |
| | heart failure. |
| Dasatinib | A tyrosine kinase inhibitor used for the treatment of lymphoblastic or |
| | chronic myeloid leukemia with resistance or intolerance to prior therapy. |
| Gefitinib | A tyrosine kinase inhibitor used as first-line therapy to treat non-small cell |
| | lung carcinoma (NSCLC) that meets certain genetic mutation criteria. |
| Nilotinib | A kinase inhibitor used for the chronic phase treatment of Chronic |
| | Myeloid Leukemia (CML) that is Philadelphia chromosome positive and for the treatment of |
| | CML that is resistant to therapy containing imatinib. |
| Lapatinib | An antineoplastic agent and tyrosine kinase inhibitor used for the |
| | treatment of advanced or metastatic HER-negative breast cancer in patients who received prior |
| | chemotherapeutic treatments. |
| Posaconazole | A triazole antifungal drug that is used to treat invasive infections by |
| | Candida species and Aspergillus species in severely immunocompromised patients. |
| Atazanavir | An antiviral protease inhibitor used in combination with other |
| | antiretrovirals for the treatment of HIV. |
| Cabazitaxel | An antineoplastic agent used for the treatment of hormone-refractory |
| | metastatic prostate cancer previously treated with a docetaxel-containing treatment regimen. |
| Temsirolimus | A antineoplastic agent used in the treatment of renal cell carcinoma (RCC) |
| | that works by inhibiting mTOR. |
| Propafenone | A Class 1C antiarrhythmic agent used in the management of paroxysmal |
| | atrial fibrillation/flutter and ventricular arrhythmias. |
| Dronedarone | An antiarrhythmic agent used in the reduce the risk of hospitalization in |
| | patients with paroxysmal or persistent atrial fibrillation. |
| Tolvaptan | A selective vasopressin V2-receptor antagonist to slow kidney function |
| | decline in patients at risk for rapidly progressing autosomal dominant polycystic kidney disease |
| | (ADPKD). Also used to treat hypervolemic and euvolemic hyponatremia. |
| Rilpivirine | A non-nucleoside reverse transcriptase inhibitor (NNRTI) used in |
| | combination with other antiretrovirals to specifically treat human immunodeficiency virus type 1 |
| | (HIV-1). |
| Boceprevir | A hepatitis C virus NS3/4A protease inhibitor used in combination with |
| | other medications to treat chronic hepatitis C genotype 1 infection. Boceprevir is not indicated as |
| | monotherapy. |
| Telaprevir | An NS3/4A viral protease inhibitor used in combination with other |
| | antivirals for the curative treatment of chronic Hepatitis C Virus infections. |
| Lomitapide | A microsomal triglyceride transfer protein inhibitor used to lower |
| | cholesterol associated with homozygous familial hypercholesterolemia (HoFH), reducing risk of |
| | cardiovascular events such as myocardial infarction and stroke. |
| Ticagrelor | A P2Y12 platelet inhibitor used in patients with a history of myocardial |
| | infarction or with acute coronary syndrome (ACS) to prevent future myocardial infarction, stroke |
| | and cardiovascular death. |
| Crizotinib | A receptor tyrosine kinase inhibitor used to treat metastatic non-small cell |
| | lung cancer (NSCLC) where the tumors have been confirmed to be anaplastic lymphoma kinase |
| | (ALK), or ROS1-positive. |
| Enzalutamide | An androgen receptor inhibitor used to treat castration-resistant prostate |
| | cancer. |
| Ivacaftor | A cystic fibrosis transmembrane conductance regulator (CFTR) |
| | potentiator used alone or in combination products to treat cystic fibrosis in patients who have |
| | specific genetic mutations that are responsive to the medication. |
| Bosutinib | An antineoplastic agent used for the treatment of chronic, accelerated, or |
| | blast phase Philadelphia chromosome-positive (Ph+) chronic myelogenous leukemia (CML) in |
| | adults with inadequate clinical response to other treatments. |
| Ponatinib | A kinase inhibitor used to treat patients with various types of chronic |
| | myeloid leukemia (CML). |
| Canagliflozin | A sodium-glucose co-transporter 2 (SGLT2) inhibitor used to manage |
| | hyperglycemia in type 2 diabetes mellitus (DM). Also used to reduce the risk of major |
| | cardiovascular events in patients with established cardiovascular disease and type 2 DM. |
| Afatinib | An antineoplastic agent used for the treatment of locally advanced or |
| | metastatic non-small cell lung cancer (NSCLC) with non-resistant EGFR mutations or resistance |
| | to platinum-based chemotherapy. |
| Simeprevir | A direct-acting antiviral agent that inhibits HCV NS3/4A protease to treat |
| | chronic hepatitis C virus (HCV) infection in adults with HCV genotype 1 or 4. |
| Idelalisib | An antineoplastic kinase inhibitor used to treat chronic lymphocytic |
| | leukemia (CLL), relapsed follicular B-cell non-Hodgkin lymphoma (FL), and relapsed small |
| | lymphocytic lymphoma (SLL). |
| Cobicistat | A CYP3A inhibitor used to increase the systemic exposure of atazanavir |
| | or darunavir in combination with other antiretroviral agents in the treatment of HIV-1 infection. |
| Rolapitant | A neurokinin-1 (NK-1) receptor antagonist used in combination with other |
| | antiemetics for the prevention of delayed nausea and vomiting associated with emetogenic |
| | chemotherapy. |
| Daclatasvir | A direct-acting antiviral agent used to treat specific hepatitis C virus |
| | (HCV) infections in combination with other antiviral agents. |
| Isavuconazonium | A triazole antifungal used for the treatment of invasive |
| | aspergillosis and mucormycosis. |
| Alectinib | A kinase inhibitor used to treat anaplastic lymphoma kinase positive |
| | metastatic non small cell lung cancer. |
| Velpatasvir | A NS5A inhibitor used to treat chronic hepatitis C infections in patients |
| | without cirrhosis or with compensated cirrhosis. |
| Lumacaftor | A protein chaperone used in combination with ivacaftor for the treatment |
| | of cystic fibrosis in patients who are homozygous for the F508del mutation in the CFTR gene. |
| Voxilaprevir | A nonstructural protein 3 and 4a protease inhibitor used to treat Hepatitis |
| | C infections. |
| Enasidenib | An isocitrate dehydrogenase-2 inhibitor used to treat relapsed or refractory |
| | acute myeloid leukemia with an isocitrate dehydrogenase-2 mutation. |
| Pibrentasvir | A Hepatitis C NS5A inhibitor used to treat Hepatitis C. |
| Glecaprevir | A Hepatitis C NS3/4A protease inhibitor used to treat Hepatitis C. |
| Abemaciclib | A medication used to treat HR+ HER2− advanced or metastatic breast |
| | cancer. |
| Candesartan | Candesartan is an angiotensin-receptor blocker (ARB) that may be used |
| | alone or with other agents to treat hypertension. It is available as a prodrug in the form of |
| | Candesartan cilexetil. |
| Letermovir | An antiviral medication used to treat CMV infections and disease in adult |
| | CMV-seropositive recipients of an allogeneic hematopoietic stem cell transplant (HSCT). |
| Rucaparib | An anti-cancer agent used to treat recurrent ovarian, fallopian tube, or |
| | peritoneal cancer. |
| Testosterone cypionate | An androgen used to treat low or absent testosterone. |
| Testosterone enanthate | An androgen used to treat low or absent testosterone. |
| Testosterone undecanoate | An androgen used to treat low or absent testosterone. |
| Isavuconazole | Indicated for patients 18 years of age and older for the treatment of |
| | invasive aspergillosis [FDA Label]. Indicated for patients 18 years of age and older for the |
| | treatment of . . . |
| Curcumin | No approved therapeutic indications. |
| Valspodar | Valspodar has been used in trials studying the treatment of Cancer, |
| | Sarcoma, Leukemia, Lymphoma, and Breast Cancer, among others. |
| Zosuquidar | Investigated for use/treatment in leukemia (myeloid) and myelodysplastic |
| | syndrome. |
| Tipranavir | A protease inhibitor used to treat HIV-1 resistant to more than 1 protease |
| | inhibitor. |
| Ranolazine | An anti-anginal drug used for the treatment of chronic angina. |
| Nelfinavir | A viral protease inhibitor used in the treatment of HIV infection. |
| Brefeldin A | A metabolite from Penicillium brefeldianum that exhibits a wide range of |
| | antibiotic activity. |
| Ceftriaxone | A broad-spectrum cephalosporin antibiotic used for the treatment of |
| | bacterial infections in various locations, such as in the respiratory tract, skin, soft tissue, and |
| | urinary tract. |
| Salinomycin | Not Annotated |
| Valinomycin | A cyclododecadepsipeptide ionophore antibiotic produced by |
| | Streptomyces fulvissimus and related to the enniatins. It is composed of 3 moles each of L- |
| | valine, D-alpha-hydroxyisovaleric acid, D-valine, and L-lactic acid linked alternately . . . |
| Bicalutamide | An androgen receptor inhibitor used to treat Stage D2 metastatic |
| | carcinoma of the prostate. |
| Mitotane | An adrenal cortex inhibitor used to treat adrenocortical tumors and |
| | Cushing's syndrome. |
| Lonafarnib | Investigated for use/treatment in solid tumors, leukemia (unspecified), and |
| | lung cancer. |
| Tipifarnib | Investigated for use/treatment in colorectal cancer, leukemia (myeloid), |
| | pancreatic cancer, and solid tumors. |
| Erlotinib | An EGFR tyrosine kinase inhibitor used to treat certain small cell lung |
| | cancers or advanced metastatic pancreatic cancers. |
| Econazole | and tinea versicolor. |
| | A topical antifungal used to treat tinea pedis, tinea cruris, tinea corporis, |
| | cutaneous candidiasis |
| Benzquinamide | Used to prevent and treat nausea and vomiting associated with |
| | anesthesia and surgery, administered intramuscularly or intravenously. |
| Tesmilifene | Intended for the treatment of various forms of cancer. |
| Ibuprofen | An NSAID and non-selective COX inhibitor used to treat mild-moderate |
| | pain, fever, and inflammation. |
| Amoxapine | A tricyclic antidepressant used in the treatment of neurotic or reactive |
| | depressive disorders and endogenous or psychotic depression. |
| Loxapine | A antipsychotic used for the treatment of schizophrenia. |
| Metronidazole | A nitroimidazole used to treat trichomoniasis, amebiasis, inflammatory |
| | lesions of rosacea, and bacterial infections, as well as prevent postoperative infections. |
| Monensin | Monensin is a polyether isolated from Streptomyces cinnamonensis that |
| | presents antibiotic properties. It is widely used in ruminant animal feeds. |
| Isradipine | A dihydropyridine calcium channel blocker used for the treatment of |
| | hypertension. |
| Niguldipine | Niguldipine is a calcium channel blocker drug (CCB) with a1-adrenergic |
| | antagonist properties. |
| Nimodipine | A calcium channel blocker used to improve neurological outcomes in |
| | patients with subarachnoid hemorrhage due to a ruptured intracranial aneurysm. |
| Yohimbine | An alpha-2-adrenergic blocker and sympatholytic found in supplements |
| | used to. |
| Toremifene | A first generation nonsteroidal selective estrogen receptor modulator used |
| | to treat certain breast cancers. |
| Laniquidar | Laniquidar has been used in trials studying the treatment of Breast Cancer. |
| Biricodar | Administered intravenously, biricodar dicitrate is to be used in |
| | combination with cancer chemotherapy agents. |
| Tariquidar | Investigated for use/treatment in ovarian cancer, lung cancer, and breast |
| | cancer. |
| Esomeprazole | A proton pump inhibitor used to treat GERD, reduce the risk of NSAID |
| | associated gastric ulcers, eradicate H. pylori, and to treat conditions causing gastric acid |
| | hypersecretion. |
| Fenofibrate | A peroxisome proliferator receptor alpha activator used to lower LDL-C, |
| | total-C, triglycerides, and Apo B, while increasing HDL-C in hypercholesterolemia, |
| | dyslipidemia, and hypertriglyceridemia. |
| Gallopamil | Gallopamil has been used in trials studying the treatment of Asthma. |
| Annamycin | Investigated for use/treatment in breast cancer and leukemia (unspecified). |
| Duloxetine | A serotonin norepinephrine reuptake inhibitor used to treat generalized |
| | anxiety disorder, neuropathic pain, osteoarthritis, and stress incontinence. |
| Ledipasvir | A direct-acting antiviral agent used to treat specific hepatitis C virus |
| | (HCV) infections in combination with other antiviral agents. |
| Paritaprevir | A direct acting antiviral agent used in combination with other antiviral |
| | agents for the treatment of Hepatitis C Virus (HCV) infections. |
| Conivaptan | An antidiuretic hormone inhibitor used to raise serum sodium levels. |
| Linagliptin | A dipeptidyl peptidase-4 (DPP-4) inhibitor used to manage hyperglycemia |
| | in patients with type 2 diabetes mellitus. |
| Suvorexant | A orexin receptor antagonist used to treat insomnia that is characterized by |
| | difficulties with sleep onset and/or sleep maintenance. |
| Vorapaxar | A platelet aggregation inhibitor used to reduce thrombotic cardiovascular |
| | events in patients with a history of myocardial infarction (MI) or peripheral arterial disease |
| | (PAD). |
| Venlafaxine | A selective serotonin and norepinephrine reuptake inhibitor (SNRI) used |
| | for the treatment of major depression. |
| Vinblastine | A vinca alkaloid used to treat breast cancer, testicular cancer, |
| | neuroblastoma, Hodgkin's and non-Hodgkins lymphoma, mycosis fungoides, histiocytosis, and |
| | Kaposi's sarcoma. |
| Propranolol | A non-selective beta adrenergic antagonist used to treat hypertension, |
| | angina, atrial fibrillation, myocardial infarction, migraine, essential tremor, hypertrophic |
| | subaortic stenosis, and pheochromocytoma. |
| Promethazine | A first-generation antihistamine used for the treatment of allergic |
| | conditions, nausea and vomiting, and motion sickness. |
| Concanamycin A | Concanamycin A is an H+-ATPase (vacuolar) inhibitor. |
| Hycanthone | Potentially toxic, but effective antischistosomal agent, it is a metabolite of |
| | LUCANTHONE. Hycanthone was approved by the FDA in 1975 but is no longer used. |
| Dexverapamil | Not Annotated |
| Emopamil | Prevents renal injury after warm & cold ischemia. |
| Lomerizine | Used to treat migraines. |
| Tetrandrine | Not Annotated |
| Dofequidar | Dofequidar is an antineoplastic agent. |
| Dexniguldipine | Not Annotated |
| ONT-093 | ONT-093 is an orally bioavailable inhibitor of P-glycoprotein (P-gp). In |
| | pre-clinical studies, ONT-093 could inhibit P-gp and reverse multidrug resistance at nM |
| | concentrations with no effect on paclitaxel pharmacokinetics. |
| Ivosidenib | An isocitrate dehydrogenase-1 inhibitor used to treat acute myeloid |
| | leukemia with a susceptible mutation. |
| Amodiaquine | For treatment of acute malarial attacks in non-immune subjects. |
| Primaquine | An antimalarial indicated to prevent relapse of vivax malaria. |
| Hydroxychloroquine | An antimalarial medication used to treat uncomplicated cases of |
| | malaria and for chemoprophylaxis in specific regions. Also a disease modifying anti-rheumatic |
| | drug (DMARD) indicated for treatment of rheumatoid arthritis and lupus erythematosus. |
| Regorafenib | A kinase inhibitor used to treat patients with metastatic colorectal cancer, |
| | unresectable, locally advanced, or metastatic gastrointestinal stromal tumors, and hepatocellular |
| | carcinoma. |
| Neratinib | A protein kinase inhibitor used to treat breast cancer that over expresses |
| | the HER2 receptor. |
| Vemurafenib | A kinase inhibitor used to treat patients with Erdheim-Chester Disease |
| | who have the BRAF V600 mutation, and melanoma in patients who have the BRAF V600E |
| | mutation. |
| Paliperidone | An atypical antipsychotic used in the treatment of schizophrenia and other |
| | schizoaffective or delusional disorders. |
| Dovitinib | Investigated for use/treatment in multiple myeloma and solid tumors. |
| Polyethylene glycol | A laxative used to treat constipation and used for bowel |
| | preparation before colonoscopies and other procedures. |
| Netupitant | An antiemetic agent used in combination with palonosetron to prevent |
| | acute and delayed vomiting and nausea caused by chemotherapy. |
| Flibanserin | A 5-HT receptor modulator used for the treatment of selected |
| | premenopausal women with acquired, generalized hypoactive sexual desire disorder (HSDD). |
| Zonisamide | A sulfonamide anticonvulsant used to treat partial seizures. |
| Venetoclax | A BCL-2 inhibitor used to treat chronic lymphocytic leukemia, small |
| | lymphocytic lymphoma, or acute myeloid leukemia. |
| Sirolimus | An immunosuppressant used to prevent organ transplant rejections and to |
| | treat lymphangioleiomyomatosis. |
| Vincristine | A vinca alkaloid used to treat acute leukemia, malignant lymphoma, |
| | Hodgkin's disease, acute erythraemia, and acute panmyelosis. |
| Doxorubicin | A medication used to treat various cancers and Kaposi's Sarcoma. |
| Zimelidine | For the treatment of depression. |
| Selegiline | A monoamine oxidase inhibitor used to treat major depressive disorder |
| | and Parkinson's. |
| Scopolamine | An anticholinergic used to treat excessive salivation, colicky abdominal |
| | pain, bradycardia, diverticulitis, IBS, and motion sickness. |
| Taurocholic acid | The product of conjugation of cholic acid with taurine. Its sodium |
| | salt is the chief ingredient of the bile of carnivorous animals. It acts as a detergent to solubilize |
| | fats. |
| Terazosin | An alpha-1 adrenergic antagonist used in the treatment of symptomatic |
| | benign prostatic hyperplasia and management of hypertension. |
| Grepafloxacin | A fluoroquinolone antibiotic used to treat various gram positive and gram |
| | negative bacterial infections. |
| Chloroquine | An antimalarial drug used to treat susceptible infections with P. vivax, |
| | P. malariae, P. ovale, and P. falciparum. It is also used for |
| | second line treatment for rheumatoid arthritis. |
| Elacridar | For the treatment of solid tumors. |
| Prednisone | A corticosteroid used to treat inflammation or immune-mediated reactions |
| | and to treat endocrine or neoplastic diseases. |
| Vandetanib | An antineoplastic kinase inhibitor used to treat symptomatic or |
| | progressive medullary thyroid cancer in patients with unresectable locally advanced or metastatic |
| | disease. |
| Sarecycline | A tetracycline antibiotic used to treat inflammatory lesions of non-nodular |
| | moderate to severe acne vulgaris. |
| Rifamycin | An antibacterial used to treat traveler's diarrhea. |
| Dacomitinib | A medication used to treat non small cell lung cancer with EGFR exon 19 |
| | deletion of exon 21 L858R substitution. |
| Tenofovir disoproxil | A nucleotide analog reverse transcriptase inhibitor used in the |
| | treatment of Hepatitis B infection and used in the management of HIV-1 infection. |
| Darunavir | A HIV protease inhibitor used in the treatment of human |
| | immunodeficiency virus (HIV) infection in patients with history of prior antiretroviral therapies. |
| Sildenafil | A phosphodiesterase inhibitor used for the treatment of erectile |
| | dysfunction. |
| Vardenafil | A phosphodiesterase 5 inhibitor used to treat erectile dysfunction. |
| Mirabegron | A beta-3 adrenergic agonist used to treat urge urinary incontinence, |
| | urinary frequency and urgency associated with overactive bladder (OAB). |
| Nefazodone | An antidepressant used in the treatment of depression. |
| Atovaquone | An antimicrobial indicated for the prevention and treatment of |
| | Pneumocystis jirovecii pneumonia (PCP) and for the prevention and treatment of |
| | Plasmodium falciparum malaria. |
| Naproxen | An NSAID used to treat rheumatoid arthritis, osteoarthritis, ankylosing |
| | spondylitis, polyarticular juvenile idiopathic arthritis, tendinitis, bursitis, acute gout, primary |
| | dysmenorrhea, and mild to moderate pain. |
| Piperine | Bioperine has been used in trials studying the treatment of Multiple |
| | Myeloma and Deglutition Disorders. |
| Asunaprevir | Asunaprevir is indicated in combination with other agents for the |
| | treatment of chronic hepatitis C in adult patients with hepatitis C virus genotypes 1 or 4 and |
| | compensated liver cirrhosis . . . |
| Glasdegib | A sonic hedgehog receptor inhibitor used to treat newly diagnosed acute |
| | myeloid leukemia in patients over 75 years who cannot receive intense chemotherapy. |
| Cetirizine | A selective Histamine-1 antagonist drug used in allergic rhinitis and |
| | chronic urticaria. |
| Palbociclib | An endocrine-based chemotherapeutic agent used in combination with |
| | other antineoplastic agents to treat HER2-negative and HR-positive advanced or metastatic |
| | breast cancer. |
| Erdafitinib | A fibroblast growth factor receptor tyrosine kinase inhibitor used to treat |
| | locally advanced or metastatic urothelial carcinoma. |
| Alpelisib | Alpelisib is indicated in combination with fulvestrant to treat |
| | postmenopausal women, and men, with advanced or metastatic breast cancer. [Label] This cancer |
| | must be hormone receptor (HR)-positive, human epidermal growth factor . . . |
| Tezacaftor | A medication used to treat homozygous or heterozygous F508del mutation |
| | cystic fibrosis. |
| Bisoprolol | A beta-1 adrenergic blocking agent used to prevent myocardial infarction |
| | and heart failure and to treat mild to moderate hypertension. |
| Entrectinib | Entrectinib is indicated for the treatment of metastatic ROS1-positive non- |
| | small cell lung cancer in adults. Entrectinib is also indicated in adults and children over 12 years |
| | old for the treatment . . . |
| Fedratinib | Fedratinib is indicated to treat adults with primary or secondary |
| | myelofibrosis that is either intermediate-2 or high risk. |
| Istradefylline | A selective adenoside A2A receptor antagonist indicated in adjunct to |
| | levodopa and carbidopa for the treatment of Parkinson's Disease. |
| Nintedanib | A triple angiokinase inhibitor indicated for the treatment of idiopathic |
| | pulmonary fibrosis, systemic sclerosis-associated interstitial lung disease, and in combination |
| | with docetaxel for non-small cell lung cancer. |
| Medroxyprogesterone acetate | A progestin used as a contraceptive and in the treatment of |
| | secondary amenorrhea, abnormal uterine bleeding, pain from endometriosis, endometrial and |
| | renal carcinomas, paraphilia in males, and GnRH-dependent precocious puberty. |
| Lasmiditan | An oral 5HT1F agonist used for the acute treatment of migraine headache |
| | with or without aura. |
| Elexacaftor | A small molecule CFTR corrector used in combination with tezacaftor and |
| | ivacaftor for the treatment of cystic fibrosis patients with one F508del-CFTR mutation. |
| Norethisterone | A synthetic second-generation progestin used for contraception, |
| | prevention of endometrial hyperplasia in hormone replacement therapy, and in the treatment of |
| | other hormone-mediated illnesses such as endometriosis. |
| Upadacitinib | An oral Janus kinase (JAK)1-selective inhibitor used in the treatment of |
| | moderate to severe rheumatoid arthritis in adult patients who did not respond well to |
| | methotrexate. |
| Cyclosporine | A steroid-sparing immunosuppressant used in organ and bone marrow |
| | transplants as well as inflammatory conditions such as ulcerative colitis, rheumatoid arthritis, and |
| | atopic dermatitis. |
| Polyethylene glycol 400 | An ingredient used in a wide variety of medications, and is |
| | not an approved medication. |
| Norgestimate | A progesterone used as a contraceptive and to treat acne vulgaris. |
| Axitinib | An oral VEGFR and kinase inhibitor used for the treatment of advanced |
| | renal cell carcinoma after failure of one prior systemic therapy. |
| Carfilzomib | A proteasome inhibitor used either alone or in conjunction with a |
| | chemotherapy regimen to treat patients with relapsed or refractory multiple myeloma. |
| Elbasvir | An antiviral and NS5A inhibitor used to treat hepatitis C infections. |
| Voacamine | For the treatment of malaria. Also under investigation for the modulation |
| | of multidrug resistance in cancer cells. |
| Elagolix | A gonadotropin releasing hormone receptor antagonist used to treat |
| | moderate to severe pain in endometriosis. |
| Olaparib | A chemotherapeutic agent used to treat recurrent or advanced ovarian |
| | cancer and metastatic breast cancer in patients with specific mutations and prior history of |
| | chemotherapy. |
| HM-30181 | Not Annotated |
| Desmethylsertraline | Desmethylsertraline is a metabolite of sertraline. |
| Reversin 121 | Reversin 121 is a hydrophobic peptide chemosensitizer that can reverse P- |
| | glycoprotein-mediated multidrug resistance. |
| Eliglustat | A glucosylceramide synthase used to treat type 1 Gaucher disease in |
| | patients who are CYP2D6 extensive, intermediate, or poor metabolizers, based on the FDA- |
| | approved test. |
| Sapropterin | A cofactor used as an adjunct to phenylalanine restriction in the treatment |
| | of phenylketonuria (PKU). |
| Everolimus | A mammalian target of rapamycin (mTOR) kinase inhibitor used to treat |
| | various types of malignancies. |
| Lamotrigine | A phenyltriazine antiepileptic used to treat some types of epilepsy and |
| | bipolar I disorder. |
| Favipiravir | An antiviral used to manage influenza, and that has the potential to target |
| | other viral infections. |
| Methylene blue | An oxidation-reduction agent used for the treatment of pediatric |
| | and adult patients with acquired methemoglobinemia. |
| Fingolimod | A sphingosine 1-phosphate receptor modulator used to treat patients with |
| | the relapsing-remitting form of multiple sclerosis (MS) and studied to manage lung |
| | complications of COVID-19. |
| Pemigatinib | A kinase inhibitor used to treat locally advanced or metastatic, |
| | unresectable cholangiocarcinoma in previously treated adult patients. |
| Ixabepilone | A microtubule inhibitor administered in combination with capecitabine or |
| | alone in the treatment of metastatic or locally advanced breast cancer that has shown inadequate |
| | response to taxanes and anthracyclines. |
| Selpercatinib | A RET receptor tyrosine kinase inhibitor for the treatment of RET-driven |
| | non-small cell lung cancer, medullary thyroid cancer, and thyroid cancer in appropriate patient |
| | populations. |
| Ripretinib | A kinase inhibitor used to treat advanced gastrointestinal stromal tumor |
| | (GIST). |
| Galantamine | A cholinesterase inhibitor used to manage mild to moderate dementia |
| | associated with Alzheimer's Disease. |
| Cethromycin | A ketolide antibiotic with broad-spectrum activity against Gram-positive, |
| | Gram-negative, and atypical bacteria that may be useful for treating several conditions including |
| | community-acquired pneumonia, inhalation anthrax, plague, and tularemia. |
| Lurbinectedin | A chemotherapeutic DNA alkylating agent used in the treatment of |
| | metastatic small-cell lung cancer. |
| Dexamethasone acetate | Commonly known as decadron, dexamethasone acetate is a |
| | glucocorticosteroid previously marketed in the USA for the treatment of inflammatory |
| | respiratory, allergic, autoimmune, and other conditions, |

Heme Oxygenase Agonists/NRF2 Agonists

Heme oxygenase-1 (HO-1), a 32 kDa enzyme containing 288 amino acid residues which is encoded by the HMOX1 gene, is a known anti-oxidant factor. HO-1 expression is induced by oxidative stress, and in animal models increasing this expression has been shown to be protective. HO-1 is found throughout the body with highest concentrations in the spleen, liver, and kidneys.

NRF2, a latent protein within each cell in the human body, is capable of turning on the production of antioxidant enzymes such as Catalase, Glutathione and Superoxide Dismutase (SOD). Once released (i.e., activated) NRF2 migrates into the cell nucleus and bonds to the DNA at the location of the Antioxidant Response Element (ARE) or also called hARE (Human Antioxidant Response Element) which is the master regulator of the total antioxidant system that is available in human cells. These antioxidant enzymes are potent neutralizers of free radicals.

Accordingly, in one embodiment, the anti-viral combination therapy of the present disclosure further comprises an HO-1 inducer (i.e., agonist), such as an NRF2 agonist. Examples of such agonists include curcumin, and other functionally similar inducers, which lower the viral replication in the cell by means of removal of cytosolic heme. In particular, curcumin, a compound of Formula III;

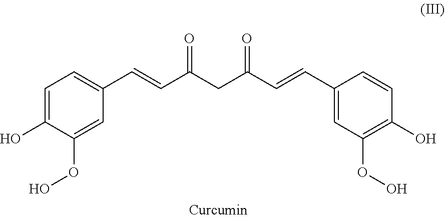 [SMILES: O=C(/C=C/c1ccc(O)c(OO)c1)CC(=O)/C=C/c1ccc(O)c(OO)c1]

is capable of removing free radicals (ROS, reactive oxygen species) in a cell (Tomeh et al., Int. J. Mol. Sci. (2019); 20(5):1033; Xu et al., Nutrients (2018) October; 10(10):1553). Curcumin impacts cellular iron metabolism, and the expression of the anti-inflammatory molecule Heme Oxygenase (HO-1), as described by Hooper (Cell Stress Chaperones. 2020 Jun. 4; 1-4. doi: 10.1007/s12192-020-01126-9). Wagener et al. described the Heme Oxygenase system as a potential target in COVID-19 patients (Antioxidants (Basel). 2020 Jun. 19; 9(6):540. doi: 10.3390/antiox9060540.). The production of HO-1 exerts multiple effects related to anti-apoptosis, anti-inflammatory, and anti-edema effects in tissues.

Curcumin is a pleiotropic molecule, with multiple cytological effects (Aggarwal and Sung, 2009). As an antioxidant, could improve cellular viability during SARS-CoV2 infection by removal of free radicals. However, N-acetyl cysteine (NAC, not shown), as well as Ascorbate (Vit C, Supp.), both failed to complement the REC combination, or substitute for curcumin. Curcumin is also a functional iron-chelator (Jiao et al. (2009). Curcumin, a cancer chemopreventive and chemotherapeutic agent, is a biologically active iron chelator. Blood), and iron metabolism is important for viral replication (Drakesmith and Prentice (2008) Viral infection and iron metabolism. Nat. Rev. Microbiol.). Clinically approved iron chelators (Deferoxamine (not shown), Deferiprone (FIG. 12)) were therefore tested but both failed to complement the REC combination or substitute for curcumin.

Additional examples of NRF2 agonists include, e.g., Bardoxolone Methyl, Dimethyl Fumarate, Omaveloxolone (RTA-408), Oltipraz, Bardoxolone, 4-Hydroxyphenylacetic acid, Sulforaphane, Obacunone, Mangiferin, Tert-butylhydroquinone (TBHQ), 4-Octyl Itaconate, Diethylmaleate, and functional equivalent compounds.

Examples of HO-1 agonists include, e.g. heavy metals, statins, paclitaxel, rapamycin, probucol, nitric oxide, sildenafil, carbon monoxide, carbon monoxide-releasing molecules, and porphyrins. Phytochemical inducers of HO include: curcumin, resveratrol, piceatannol, caffeic acid phenethyl ester, dimethyl fumarate, fumaric acid esters, flavonoids, chalcones, Ginkgo biloba, anthrocyanins, phlorotannins, camosol, rosolic acid, and numerous other natural products. Endogenous inducers include i) lipids such as lipoxin and epoxyeicosatrienoic acid; and ii) peptides such as adrenomedullin and apolipoprotein; and iii) hemin. NRF2 inducers with downstream HO-1 induction include: genistein, 3-hydroxycoumarin, oleanolic acid, isoliquiritigenin, PEITC, diallyl trisulfide, oltipraz, benfotiamine, auranofin, acetaminophen, nimesulide, paraquat, ethoxyquin, diesel exhaust particles, silica, nanotubes, 15-deoxy-Δ12,14 prostaglandin J2, nitro-oleic acid, hydrogen peroxide, and succinylacetone.

### Pharmaceutical Compositions

In another aspect, the present invention provides a composition or combination, e.g., a pharmaceutical composition or separately administered combination, of active agents, formulated with a pharmaceutically acceptable carrier, e.g., each component of the combination can be formulated separately or together with the carrier.

Pharmaceutical compositions of the invention also can be administered in combination therapy, i.e., combined with other agents. For example, the combination therapy can include a combination or composition of the present invention combined with at least one other therapeutic agent, e.g., a corticosteroid, an anti-inflammatory signal transduction modulator, a β2-adrenoreceptor agonist bronchodilator, an anticholinergic, amucolytic agent, hypertonic saline, and other drugs for treating a virus infection. For example, in one embodiment the other active therapeutic agent is active against Corona virus infections. In another embodiment the other active therapeutic agent is active against Arenaviridae virus infections, particularly Lassa virus and Juninvirus infections. Non-limiting examples of these other active therapeutic agents are ribavirin, favipiravir (also known as T-705 or Avigan), T-705 monophosphate, T-705 diphosphate, T-705 triphosphate, ST-193, and mixtures thereof. The combinations and compositions of the present invention are also intended for use with general care provided patients with viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or anti-diarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for oral, inhalation, intravenous, intramuscular, subcutaneous, parenteral, spinal or epidermal administration (e.g., by injection or infusion). Depending on the route of administration, the active compound, i.e., the combination or composition, may be coated in a material to protect the compound from the action of acids and other natural conditions that may inactivate the compound.

The pharmaceutical compounds of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For clinical administration of the combinations and compositions, the dosage can range from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months. Preferred dosage regimens of the invention include 1 mg/kg body weight or 3 mg/kg body weight via intravenous administration, with the combination or composition being given using one of the following dosing schedules: (i) every four weeks for six dosages, then every three months; (ii) every three weeks; (iii) 3 mg/kg body weight once followed by 1 mg/kg body weight every three weeks. Intervals between single dosages can be, for example, weekly, monthly, every three months or yearly.

In a particular embodiment, the ABCB1/ABCG2 inhibitor is administered at about 100 mg to 200 mg, remdesivir is administered at about 20 mg to 300 mg (e.g., 50 mg to 300 mg), and the optional NRF2 agonist is administered at about 100 mg to 1,000 mg, e.g., once daily at about 500 to 25 mg/kg.

In another embodiment:
(a) ABCB1/ABCG2 inhibitor is administered in a dosage amount of about 50 mg to 300 mg.
(b) remdesivir inhibitor is administered in a dosage amount of about 100 mg to about 200, and
(c) the optional NRF2 agonist is administered in a dosage amount of about 100 mg to about 1000 mg.

In a particular embodiment, the ABCB1/ABCG2 inhibitor (e.g., elacridar, tariquidar, or zosuquidar) is administered before administration of remdesivir. For example, in one aspect, administration of tariquidar prior to administration of remdesivir results in ABC-inhibition prior to administration of remdesivir, thus maximizing the effective interference of cytoplasmic export of remdesivir (i.e., reducing the loss of remdesivir).

Alternatively, the combination or composition can be administered as a sustained release formulation, in which case less frequent administration is required. Dosage and frequency vary depending on the half-life of the active ingredient. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of a combination or composition of the invention preferably results in a decrease in severity of the viral infection symptoms, an increase in frequency and duration of infection symptom-free periods, or a prevention of impairment or disability due to the infection. One of ordinary skill in the art would be able to determine such amounts based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. A preferred route of administration for combinations or compositions of the invention includes oral administration.

Other routes of administration include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Alternatively, a combination or composition of the invention can be administered via a non-parenteral route, such as a topical, epidermal or mucosal route of administration, for example, intranasally (i.e., by inhalation), vaginally, rectally, sublingually or topically. For example, a combination or composition of the present invention can be administered to a subject by way of the lung. Pulmonary drug delivery may be achieved by inhalation, and administration by inhalation herein may be oral and/or nasal. Examples of pharmaceutical devices for pulmonary delivery include metered dose inhalers, dry powder inhalers (DPIs), and nebulizers. For example, a composition described herein can be administered to the lungs of a subject by way of a dry powder inhaler. These inhalers are propellant-free devices that deliver dispersible and stable dry powder formulations to the lungs. Dry powder inhalers are well known in the art of medicine and include, without limitation: the TurboHaler® (AstraZeneca; London, England); the AIR® inhaler (Alkermes®; Cambridge, Mass.); Rotahaler® (GlaxoSmithKline; London, England); and Eclipse™ (Sanofi-Aventis; Paris, France). See also, e.g., PCT Publication Nos. WO 04/026380, WO 04/024156, and WO 01/78693. DPI devices have been used for pulmonary administration of polypeptides such as insulin and growth hormone. A composition described herein can be intrapulmonarily administered by way of a metered dose inhaler. These inhalers rely on a propellant to deliver a discrete dose of a compound to the lungs.

A composition described herein can be administered to the lungs of a subject by way of a nebulizer. Nebulizers use compressed air to deliver a compound as a liquefied aerosol or mist. A nebulizer can be, e.g., a jet nebulizer (e.g., air or liquid-jet nebulizers) or an ultrasonic nebulizer. Additional devices and intrapulmonary administration methods are set forth in, e.g., U.S. Patent Application Publication Nos. 20050271660 and 20090110679.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, combinations and compositions of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153: 1038); antibodies (P. G. Bloeman et al. (1995) FEBS Lett. 57:140; M. Owais et al. (1995) Antimicrob. Agents Chemother. 9:180); surfactant protein A receptor (Briscoe et al. (1995) Am. J. Physiol. 1233:134); p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273.

Uses of the Invention

The combinations and compositions of the present invention have multiple anti-viral in vitro and in vivo diagnostic and therapeutic utilities. For example, these molecules can be administered to cells in culture, e.g. in vitro or ex vivo, or in a subject, e.g., in vivo, to treat, prevent or diagnose a viral infection. The term “subject” as used herein in intended to includes human and non-human animals. Non-human animals includes all vertebrates, e.g., mammals and non-mammals, such as non-human primates, sheep, dogs, cats, cows, horses, chickens, amphibians, and reptiles. The methods are particularly suitable for treating human patients having a virus infection. When combinations of the active ingredients are administered, they can be administered in any order or simultaneously.

Also within the scope of the invention are kits comprising the combinations or compositions of the invention and instructions for use. The kit can further contain a least one additional reagent. Kits typically include a label indicating the intended use of the contents of the kit. The term label includes any writing, or recorded material supplied on or with the kit, or which otherwise accompanies the kit.

The present disclosure is further illustrated by the following examples, which should not be construed as further limiting. The contents of all figures and all references, Genbank sequences, patents, and published patent applications cited throughout this application are expressly incorporated herein by reference.

### EXAMPLES

### Example 1: High Dimensional Design of Experiments Addressing Combinatorial Spaces Among Known Drugs

Experimental Design

Rapid development of interventions that may provide clinical efficacy against emerging viral pandemics is relevant, and in the case of SARS-CoV2 critical. To this end, multiple avenues of research have been explored, including extensive machine-based bioinformatics-driven approaches, high-throughput drug screening, re-purposing of existing drugs, and accelerated development of vaccines. While increasingly powered by the rapid growth of data, bioinformatics-based approaches need to be complemented by empirical testing, as hits emerge through prediction-based scoring. High-throughput drug screening is a powerful method to identify novel drugs, especially when a target is identified, but its utility is less effective when the goal is interference with the lifecycle of a virus, such as SARS-CoV2. Singular drugs that have proven highly effective against other viruses are rare. Progress in vaccine development is rapid, and record-breaking, yet it remains unknown if vaccination against SARS-CoV2 may provide lasting immunity.

Accordingly, in this Example, an HD DoE assay to detect the combinatorial effects of pharmacological compounds was performed as described in Esakov et. al, Endocrine Related Cancer, https://pubmed.ncbi.nlm.nih.gov/31167163/. A composite design was generated in which compounds (i.e., “input drugs”) were administered in combinations through an experimental design constructed based on mathematical algorithms. A master plate of stock compounds (obtained from ApexBio®) was created in which drugs were provided a mM-concentration. A dilution, and reconfiguring, process was executed robotically to create a series of secondary master plates, consisting of drugs in which individual wells contained specific combinations of drugs. This secondary master plate was further diluted to final assay concentrations, which was provided to cells, either in absence (for toxicity screening), or presence (for anti-viral effects) of the SARS-CoV-2 virus. The effect of the combinations of the drugs was measured in the SARS-CoV-2 assay, and the data (relating to survival of cells) was treated as separate response variables. The SARS-CoV-2 assay included the following steps: (a) the cells were seeded (e.g., 25.000 cells are seeded in 100 μl of assay medium and incubated overnight to allow the cells to settle on the bottom and adhere); (b) the next day, the cells are treated and infected, e.g., with 50 μl of input compounds; (c) after 2-3 hours, 50 μl of virus is added to infect the cells (MOI of ˜0.004); and (d) assays were read on days 1-4.

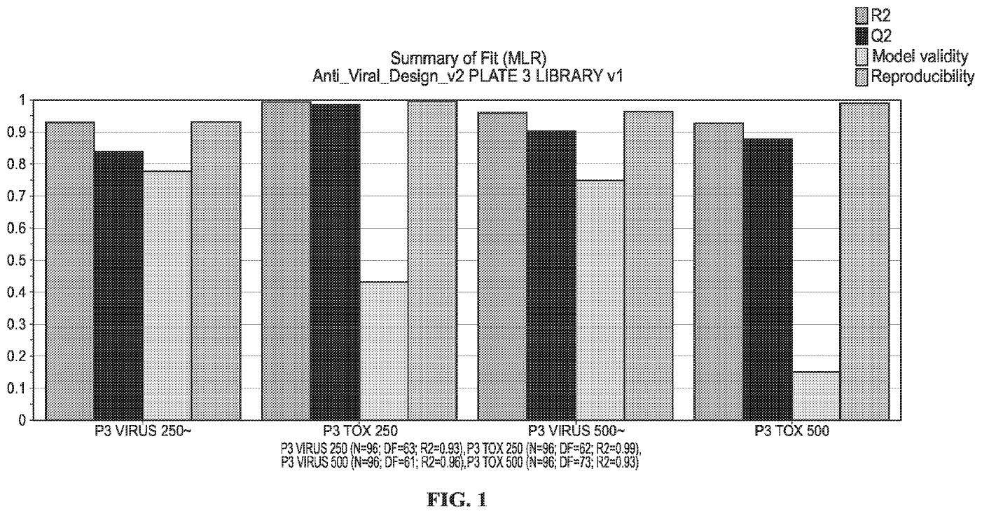

The outcome data based on the response variables (measured as integrated fluorescence related to the EGFP-fluorescence) was mathematically fitted against the effector (i.e., the input drugs) using the mathematical fit method of MLR (multiple linear regression fit). The results are shown in FIG. 1 as an effector/response matrix that contains statistical information of any possible first-level interactions that have existed between any of the tested input drugs. If multiple interactions terms existed between more than two drugs, secondary interaction terms were added, to explain triple interaction synergy. The outcome response variance was explained by the R2 value. An R2 value of >0.2 signifies a useful model. The predictive power of the model obtained was evaluated by the Q2 measurement, where a Q2 value>0.1 signifies a model that provides predictive strength. The reproducibility was calculated based on in-design centered control points, with no drugs added (quadruplicate).

Imaging scans of VERO-EGPF cells across a high-dimensional design of experiments plate/Toxicology testing were obtained (data not shown) to illustrate the underlying process of obtaining the response data. Each cell of an initial plate was seeded with an equal amount of EGFP-fluorescing VERO-EGFP cells. Twelve independent drugs were administered in combination across the entire plate, and the plate was scanned for fluorescence intensity 4 days later. Specifically, high-content imaging of cellular fluorescence was converted into response data and subsequently fitted onto the design of experiments-based administration of the individual drugs. The response data were converted into specific models based on the toxicology data related to a loss of cells (i.e., death of cells) caused by addition of the drug Brefeldin A. The coefficient plots were generated for all responses and qualitatively inspected by the R2 and Q2 parameters.

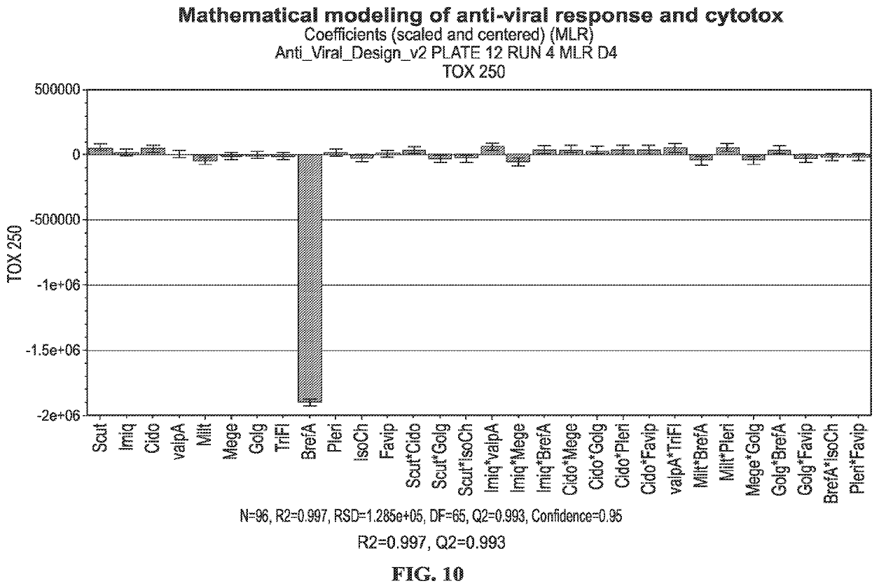

A coefficient plot of these data (FIG. 10) shows toxicity correlates to loss of fluorescence intensity. The pattern of cell loss was attributable to the presence of particular drugs, as administered through the HD-DoE design. The scan data was fitted onto the experimental design, and mathematical modeling was performed. The observed variance was explained by the model with a quality related to the R2 value (here 0.997). Following model tuning for maximizing the predictive power (Q2, here 0.993) a complex model emerges, from which the system response is plotted. As shown, the drug BrefA (Brefeldin A) is cytotoxic and the cause of the observed cell loss (i.e., cell death). Other terms in the model are needed to adequately explain the complex response to the twelve drugs, as indicated by the number of primary and secondary mathematical terms. Each starred term represents an interaction term between two distinct drugs.

In the present invention, >250 distinct antiviral drugs were tested, first in groups of 12 to identify plausible drug/drug interactions which favored interference with the viral cytopathic response. In the first screen, 20 combinatorial plates were analyzed. Subsequent screens focusing on the emerging hits were subsequently performed to validate and, if possible, improve upon any identified combinations. The REC combination was determined from one of the initial plates, wherein 12 separate and distinct compounds where tested for synergistic effects against SARS-CoV-2 as follows: Irbesartan, clemizole, clemizoleHCL, curcumin, elacridar, flavopiridol, GS-9620, camostat, letermovir, remdesivir, trigonelline, and favipiravir, and is described in more detail here.

Using the SARS-CoV-2 assay read out, VERO-EGFP cells were seeded and inoculated with virus on Day 1 after seeding. The input compounds were added to the secondary master plate at designated concentrations (i.e., 250 nM and 500 nM). The EGFP fluorescence (an indicator of cell survival) was measured for four days. In parallel, data for cell survival (Tox) of cells without virus, but with input compounds, was similarly obtained.

Based on the data collected at day four, the mathematical fit of both Tox and Antiviral responses were strong. Logarithmic transformation of the antiviral response data was performed to attain the strongest model. The observed R2 value for virus response at 250 nM was 0.93, Q2=0.84, model validity 0.78, and reproducibility 0.94. These values indicated a very strong approximation of the response space, explaining 93% of the observed variance, and returned an exceptionally strong goodness of fit at 0.78. The values for the corresponding Tox and Viral response at 500 nM is shown in FIG. 1 which shows strong mathematical modeling of the responses.

In conclusion, the data provided herein and below show that the HD-DoE method, when segmented, can be rapidly employed to explore the combination space among known drugs for attaining effective control of viral replication in a relevant mammalian cell. The REC combination provides significant clinical intervention in COVID-19 for both recently diagnosed patients as well patients progressing and experiencing high viral burdens.

### Example 2: Triple Synergism Among Remdesivir, Elacridar, and Curcumin Strongly Alleviates SARS-CoV2 Cytopathic Effects in Vero-EGFP Cells

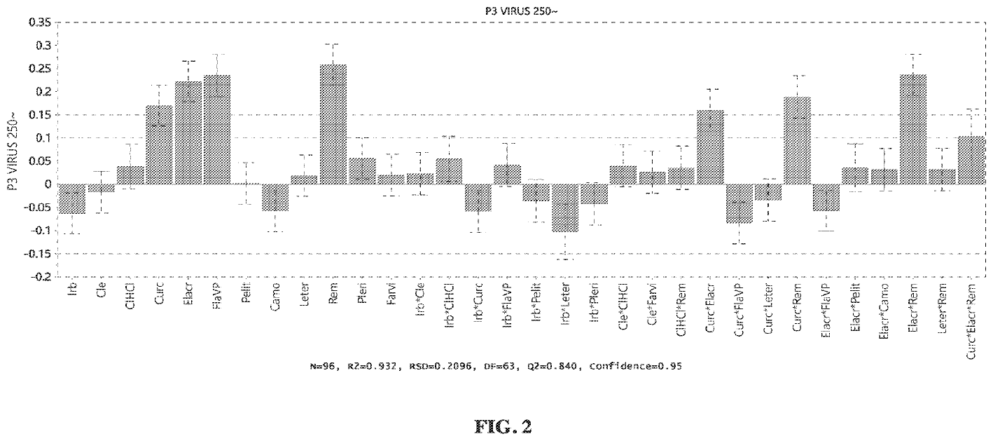

The mathematical modeling of the response data described in Example 1 was tuned to attain maximal Q2 (predictive power) as shown in FIG. 2. As shown in FIG. 2, two compounds (irbesartan and camostat) negatively impacted the survival of the cells in response to the SARS-CoV-2 virus. In contrast, an antiviral response was demonstrated by four drugs: elacridar, curcumin, remdesivir, and flavopiridol. Notably, statistically significant terms (detected within 95% confidence intervals) were detected between (a) elacridar and curcumin, (b) curcumin and remdesivir, and (c) remdesivir and elacridar. Flavopiridol did not interact significantly with the other three compounds.

Synergism among three compounds (remdesivir, elacridar, and curcumin) also was demonstrated. Specifically, inclusion of the triple-interaction term of “Curc*Elac*Rem” in the model signified the existence of triple synergism among these compounds and improved the Q2 value. The triple interaction term also was detected within a 95% confidence interval and kept for further analysis of the model.

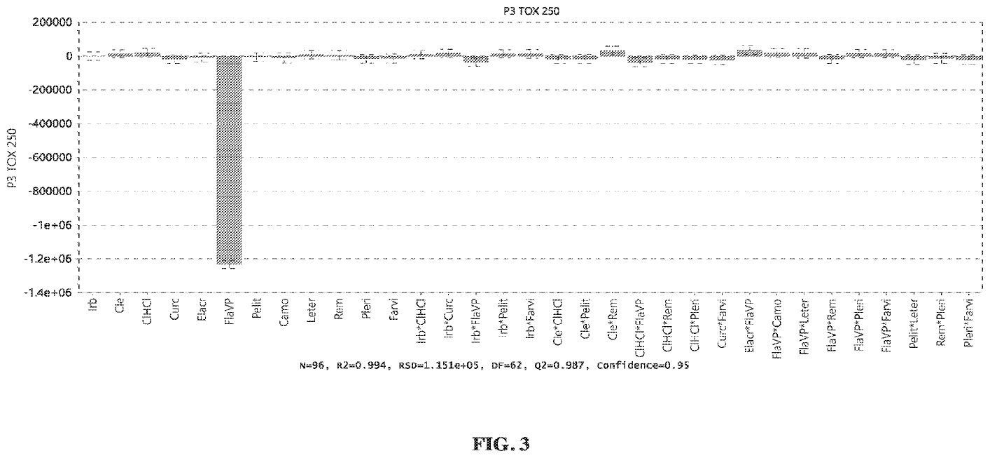

The corresponding data for toxicology (as shown in FIG. 3) showed that Flavopiridol individually caused cell death. The effector term for this compound is essentially absolute and therefore this compound did not interact significantly with other compounds in the screen for its toxicity. In parallel, it can be concluded that none of the three drugs in the triad of remdesivir, elacridar, and curcumin negatively impacted cell viability, either alone or within their respective combinations.

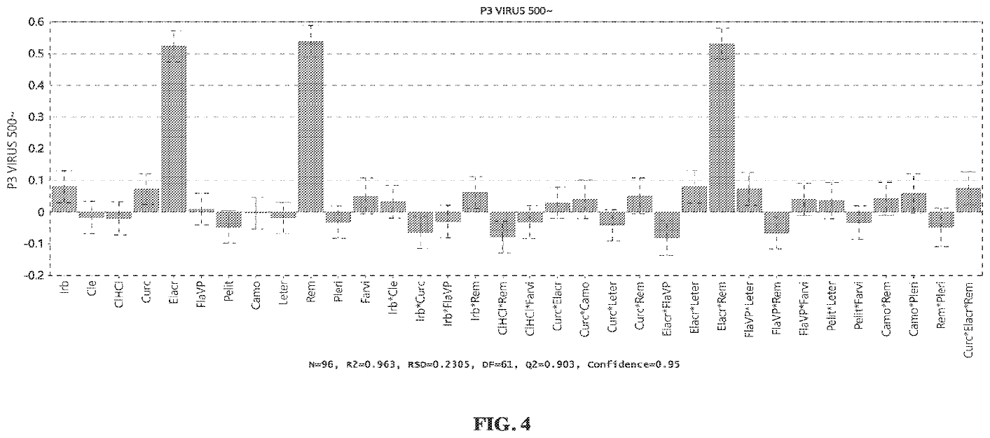

When performing the same analysis above, but increasing the total amount of input drug to 500 nM, the antiviral response changed to reflect that the relevance of curcumin is less important, i.e., strong synergism still exists between remdesivir and elacridar, and the data supports antiviral activity based on the triple interaction term of Curc*Elac*Rem. Therefore, curcumin is still shown to be potently enhancing the other two, yet with less significance than if the drugs were applied at lower concentration. Corresponding toxicology analysis at the higher concentration of 500 nM (as shown in FIG. 4) confirmed the previous finding that Flavopiridol is cytotoxic, and that curcumin and remdesivir had no cytotoxicity.

### Example 3: Criticality Analysis of Triple Combination Anti-Viral Therapy

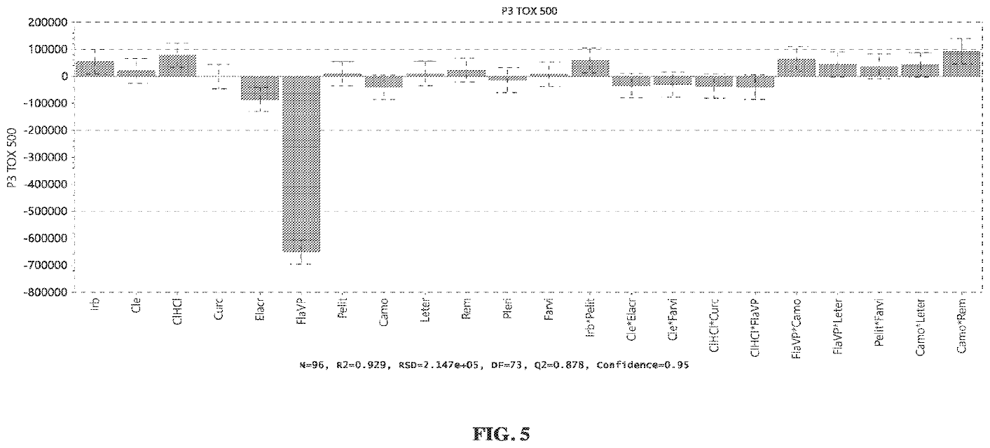

Based on the mathematical model of the response space, it was possible to perform in-silico analysis of the factor effects (attributed to the triple combination therapy of remdesivir, elacridar, and curcumin), and their relative criticality. This process is entirely data driven and based on statistical analysis. Creating the conditions, in which all irrelevant drugs in the screen are set to zero, the system response is shown in FIG. 5 through a setpoint analysis where the three drugs (remdesivir, elacridar and curcumin) are provided. The relative importance of the three drugs are shown from left to right, revealing the order of remdesivir, elacridar and curcumin. White bands refer to 95% confidence interval bands. As can be seen, all three drugs positively impacted the ability of the Vero-EGFP cells to survive following SARS-CoV2 infection. Criticality testing of each factor can now be performed by individually removing each drug from the mix.

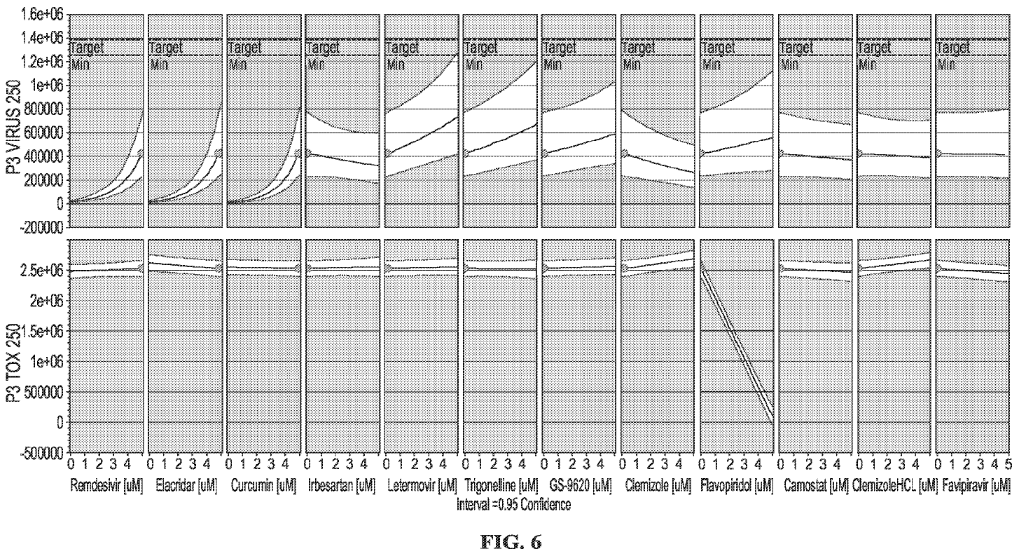

In FIG. 6, the setpoint was modified wherein curcumin was removed, and neither elacridar nor remdesivir were able to inhibit the viral effect at the specified concentrations. Specifically, FIG. 6 shows a plot of the system response for the condition of added remdesivir (R), elacridar (E) and curcumin (C) top row, P3 (plate 3), VIRUS, factors provided each at 250 nM concentration final (X-axis lists stock concentrations in experiments). All other inputs are set to zero. White space indicates 95% confidence intervals. The order of factors in relation to induction of maximal suppression of cytopathic response of SARS-CoV-2 is listed in decreasing order from left, to right. R is most critical, E subsequently, and C, hereafter. The maximal blockade of the cytopathic effect of the SARS-CoV2 virus on Vero-EGFP cells were obtained when all three compounds are administered simultaneously (R+E+C).

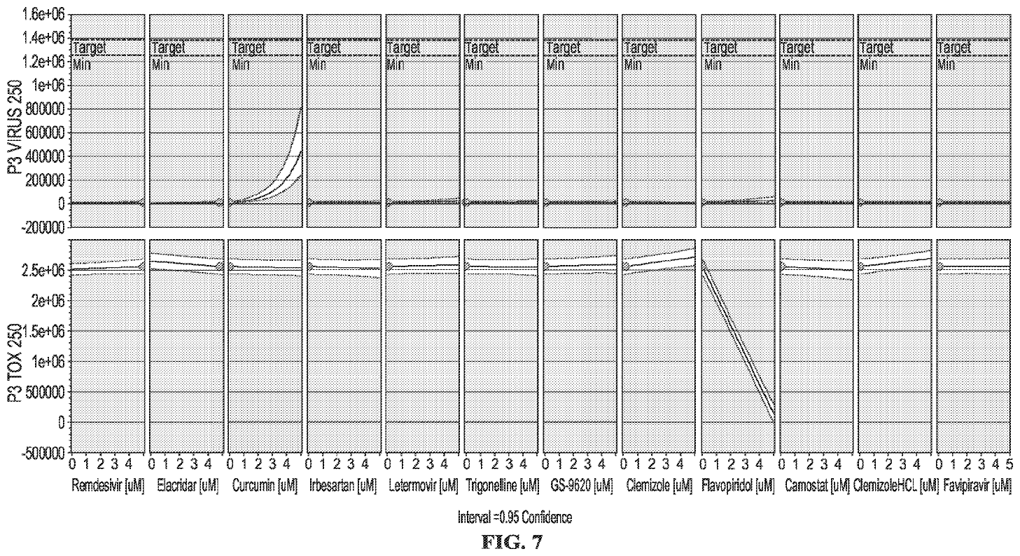

In FIG. 7, the setpoint was modified wherein curcumin was removed, and neither elacridar nor remdesivir were able to inhibit the viral effect at the specified low concentrations. FIG. 7 shows a plot of the system response for the condition of added remdesivir (R), elacridar (E) and curcumin (C) top row, P3 (plate 3), VIRUS, factors provided each at 250 nM concentration final (X-axis lists stock concentrations in experiments). All other inputs are set to zero. White space indicates 95% confidence intervals. The order of factors in relation to induction of maximal suppression of cytopathic response of SARS-CoV-2 is listed in decreasing order from left, to right. R is most critical, E subsequently, and C, hereafter. In this plot, curcumin was removed from the recipe. The resulting effect of the response was complete abrogation of protection from virus mediated cytopathic effects (notice the flat response for R, E). Inspection of cytotoxicity of the compounds (when virus is absent) is shown in second row (P3 TOX 250 nM). None of the R, E, C compounds displayed cytotoxic effects. However, the compound flavopiridol is highly toxic to the Vero-EGFP cells.

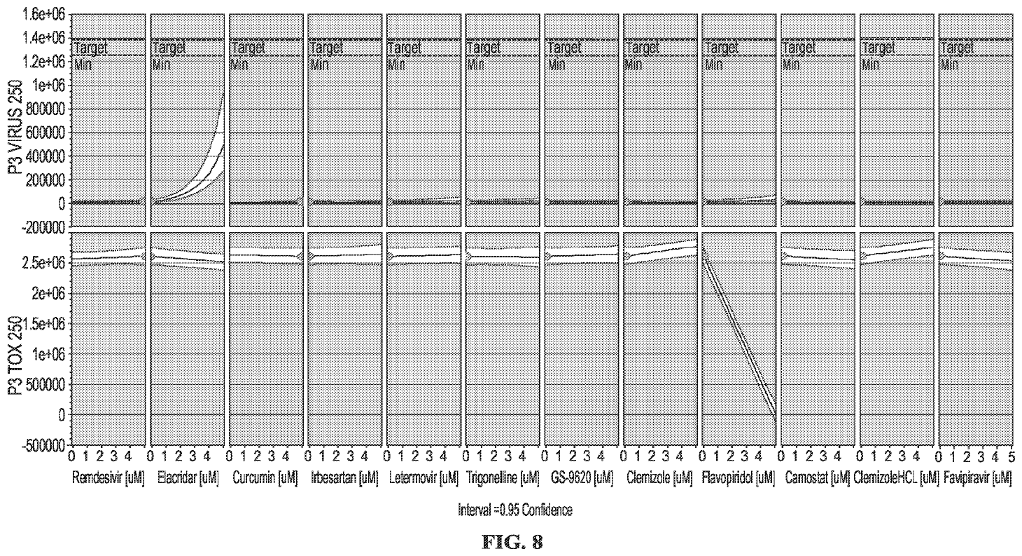

At higher concentrations of elacridar and remdesivir, however, curcumin was not needed to inhibit the viral effect. In FIG. 8, elacridar was removed, and neither remdesivir nor curcumin were able to inhibit the viral effect. FIG. 8 shows a plot of the system response for the condition of added remdesivir (R), elacridar (E) and curcumin (C) top row, P3 (plate 3), VIRUS, factors provided each at 250 nM concentration final (X-axis lists stock concentrations in experiments). All other inputs are set to zero. White space indicates 95% confidence intervals. The order of factors in relation to induction of maximal suppression of cytopathic response of SARS-CoV-2 is listed in decreasing order from left, to right. R is most critical, E subsequently, and C, hereafter. In this plot, elacridar was removed from the recipe. The resulting effect of the response was complete abrogation of protection from virus mediated cytopathic effects (notice the flat response for R, C).

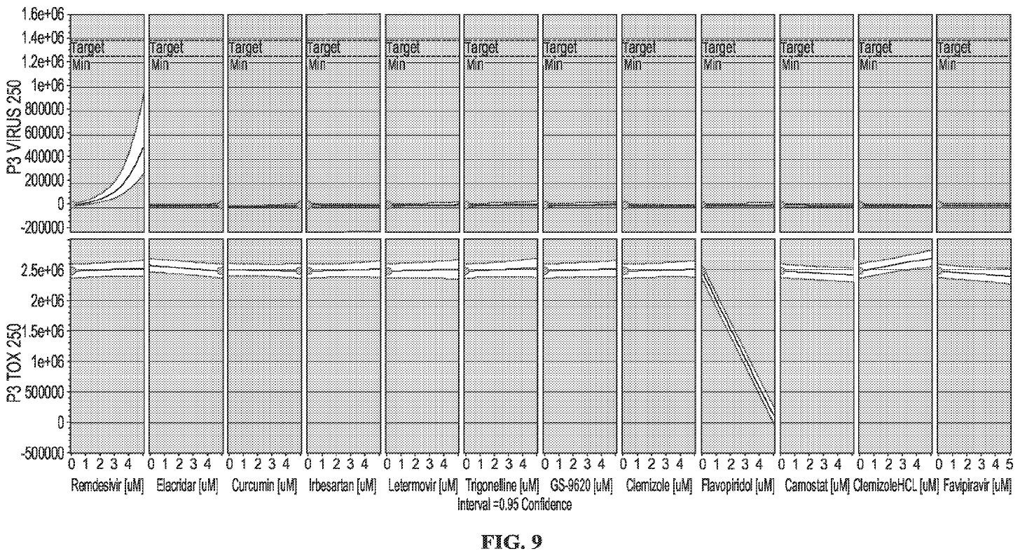

In FIG. 9, remdesivir was removed and neither elacridar nor curcumin were able to inhibit the viral effect. FIG. 9 shows a plot of the system response for the condition of added remdesivir (R), elacridar (E) and curcumin (C) top row, P3 (plate 3), VIRUS, factors provided each at 250 nM concentration final (X-axis lists stock concentrations in experiments). All other inputs are set to zero. White space indicates 95% confidence intervals. The order of factors in relation to induction of maximal suppression of cytopathic response of SARS-CoV-2 is listed in decreasing order from left, to right. R is most critical, E subsequently, and C, hereafter. In this plot, remdesivir was removed from the recipe. The resulting effect of the response was complete abrogation of protection from virus mediated cytopathic effects (notice the flat response for E, C).

Altogether, the data show strong triple synergism and independent criticality of each of the compounds in this combination at the specified concentrations. In FIG. 7, the setpoint was modified wherein curcumin was removed and neither elacridar nor remdesivir were able to inhibit the viral effect at the specified low concentrations. At higher concentrations of elacridar and remdesivir, however, curcumin was not needed to inhibit the viral effect. In FIG. 8, elacridar was removed and neither remdesivir nor curcumin were able to inhibit the viral effect. In FIG. 9, remdesivir was removed, and neither elacridar nor curcumin were able to inhibit the viral effect.

Three-dimensional plots were created which visualize the synergistic effects of the compounds according to the two-way interactions (data not shown). These three-dimensional plots were created with all other drugs set to zero and the components in the combination not plotted set at maximal. For example, one plot showed the response to elacridar and curcumin, with 250 nM of remdesivir added and Vero-EGFP cells/4 days exposure to SARS-CoV-2 virus. This plot exhibited a sloped response plane which correlates to synergism. Maximum was attainable at max concentration of either compound. A second plot showed the response to elacridar and remdesivir, with 250 nM of curcumin added and Vero-EGFP cells/4 days exposure to SARS-CoV-2 virus. This plot also exhibited a sloped response plane which correlates to synergism. Maximum was attainable at max concentration of either compound. A third plot showed the response to curcumin and remdesivir, with 250 nM of elacridar added and Vero-EGFP cells/4 days exposure to SARS-CoV-2 virus. Like the other two plots, this plot exhibited a sloped response plane is shown which correlates to synergism. Maximum was attainable at max concentration of either compound.

Accordingly, as discussed above, the particular combination of the ribonucleoside analogue remdesivir, the ABCB1/ABCG2 dual inhibitor elacridar, and optionally curcumin, was highly effective in blocking the viral lethality of the SARS-CoV2 host cell. As shown, the potency of the combination was remarkable and able to completely abolish viral cytopathy below 70 nM of administration of the compounds. Moreover, as further discussed below, more specific inhibitors of either ABCB1 or ABCG2 alone were unable to substitute for elacridar in the combination. However, the known dual-specificity inhibitor tariquidar was able to substitute for elacridar, indicating that remdesivir is exported by both ABCB1 and ABCG2, and therefore that effective interference with cytoplasmic export requires a dual-specificity inhibitor of the elacridar/tariquidar type.

Effective interference of SARS-CoV2 cytopathy was also achieved using remdesivir and elacridar alone. A gradual reduction in the relevance of curcumin at higher concentrations of remdesivir/elacridar was observed. However, at lower concentrations, the importance of curcumin was noted.

### Example 4: Favirpiravir does not Impact the Effect of the REC Combination

The specificity of the identified combination of remdesivir, elacridar, and curcumin (R+E+C) is further supported by the observed lack of interaction the combination exhibits with another compound, favipiravir, which has been reported to have efficacy against SARS-CoV2. As shown in a 4-dimensional plot (data not shown), favipiravir had no effect on the efficacy of the remdesivir, elacridar, and curcumin combination. Specifically, the four-dimensional plot included remdesivir, elacridar, curcumin and farvipiravir in Vero-EGFP cells/4 days exposure to SARS-CoV-2 virus. This plot showed the inhibitory effect of the triple combination effect and that the farvipiravir compound did not impact the effect of the R+E+C combination.

### Example 5: Efficacy of Dual ABCB1 (P-Glycoprotein)/ABCG2 Pathway Iinhibitors in REC Combination Therapy

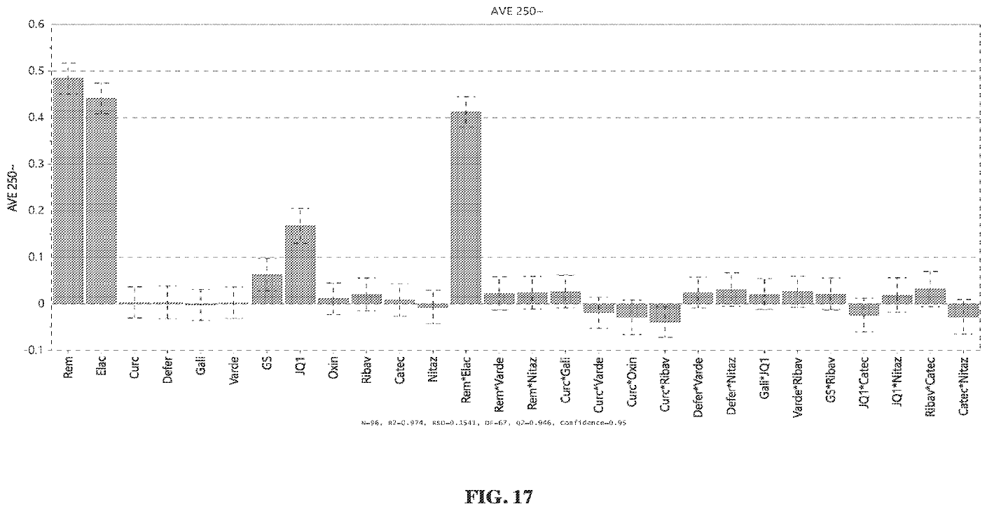

Focus testing of the REC combination was performed using known ABC-family inhibitors. As shown in FIG. 11, febuxostat and K0143 cannot substitute for elacridar in the REC combination, i.e., neither of these drugs impacted the REC combination. Both drugs are known ABCG2 inhibitors. Therefore, the data show that ABCG2 inhibitors can be excluded as the target for the mechanism of action of elacridar. High content image scans were obtained as a representation of actual scan data for the testing of elacridar substitutes, tariquidar and zosuquidar (Toxicology and antiviral effect at 250 and 500 nM) (scans not shown). As demonstrated by these scans, the effective abolishment of the cytopathic effect was achieved in approximately half of the wells, regardless of concentration. The coefficient plot is shown in FIG. 12 which demonstrated that tariquidar and zosuquidar each have the same effect as elacridar and can be substituted for elacridar, i.e., compared to elacridar, either compound is equally potent in synergizing with remdesivir. All three compounds are known inhibitors of ABCB1 (P-glycoprotein, MDR), accordingly, the data show that the mechanism of action of elacridar is through the P-glycoprotein pathway inhibition. To further determine the specificity of the mechanism of action of elacridar, the ABCC1 inhibitor vardenafil (Varde) was also tested (FIG. 17). Unlike tariquidar and zosuquidar, the ABCC1 inhibitor does not have the same effect as elacridar (i.e., it has no effect on the REC combination) and cannot be substituted for elacridar.

The specificity of elacridar to remdesivir export was determined through additional testing. Inhibition of ABCG2 using the ABCG2-specific inhibitor Ko143 failed to complement for elacridar (FIG. 11). Furthermore, febuxostat, another inhibitor of ABCG2, failed to complement (FIG. 11). However, tariquidar, a low-nM dual-specificity ABCB1 (5.1 nM)/ABCG2 inhibitor substituted for elacridar (FIG. 12), as did zosuquidar. As shown in FIG. 12, equal antiviral potency (as measured in a statistically significant manner with, i.e., within 95% confidence) was observed between (a) remdesivir (REM) and elacridar (ELAC), (b) remdesivir (REM) and tariquidar (TARIQ), (c) remdesivir (REM) and zosuqidar (ZOSUQ). The negative interaction terms between ELAC*TARIQ and ELAC*ZOSUQ, and TARIQ*ZOSUQ demonstrate that these drugs are not additive. Also shown, EIDD1 (EIDD1931) does not exert an antiviral effect.

Quercetin is a known ABCB1 inhibitor but operates at a higher concentration than elacridar/tariquidar/zosuquidar. It failed to complement for elacridar (FIG. 11). Similarly, ONT-093 is specific for ABCB1 over ABCG2, and failed to complement (FIG. 12). Vardenafil was also found to be a potent inhibitor of ABCB1 (Ding et al. (2011). The phosphodiesterase-5 inhibitor vardenafil is a potent inhibitor of ABCB1/P-glycoprotein transporter. PLoS One) and also failed to complement elacridar (data not shown). Accordingly, dual inhibition of ABCG2/ABCB1 effectively abrogated remdesivir drug efflux which can be achieved using dual-specificity inhibitors of the ABC-family.

### Example 6: Remdesivir is a Potent Nucleoside Inhibitor in the REC Combination

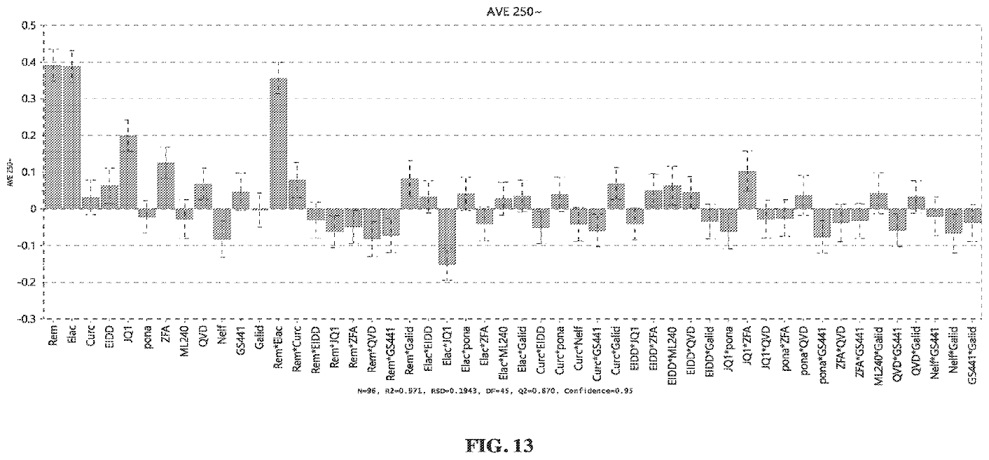

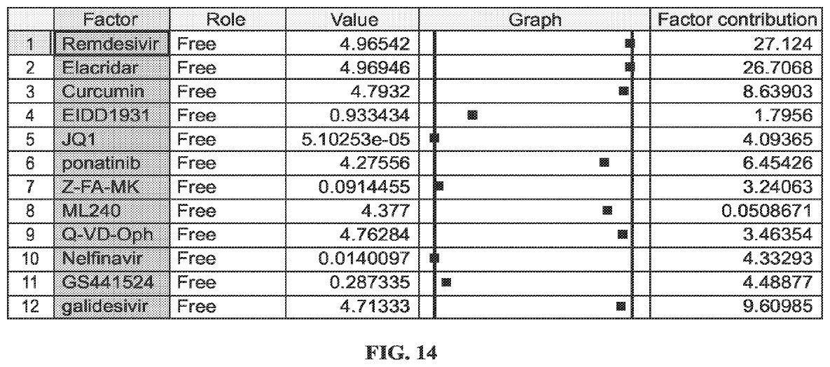

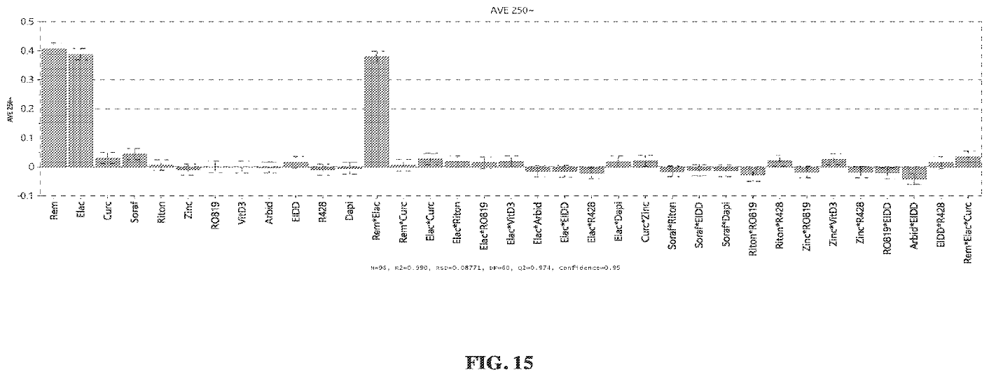

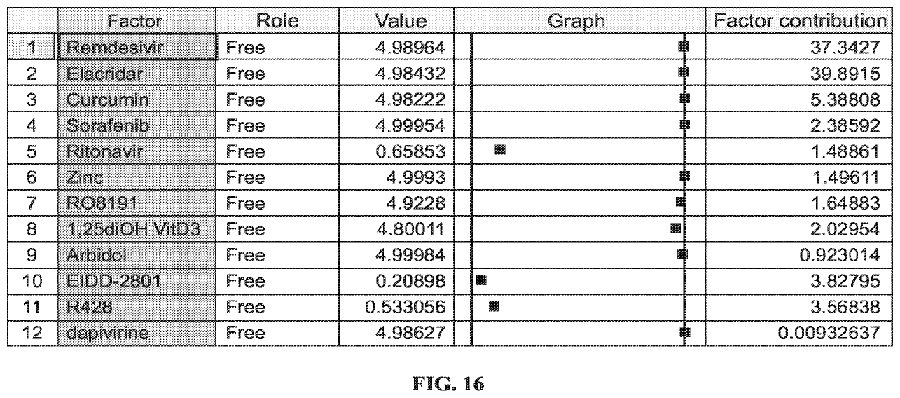

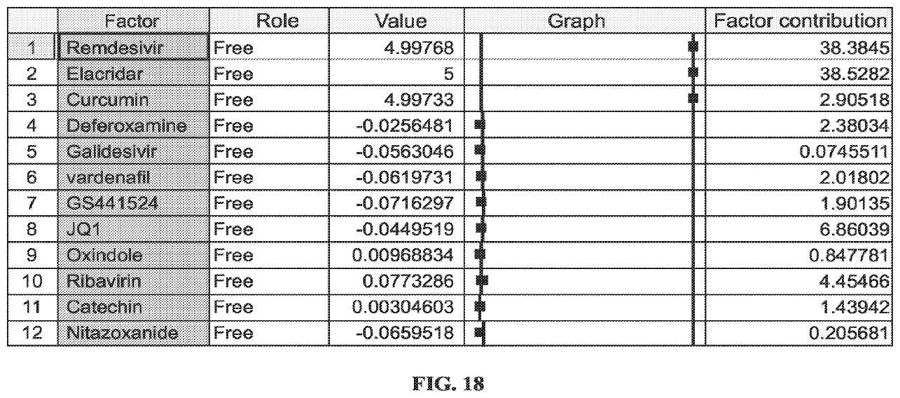

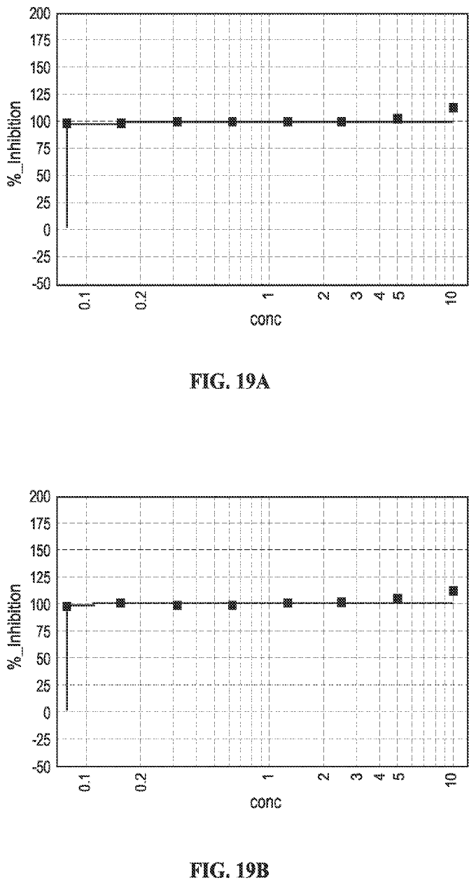

Focus testing of the REC combination was performed to identify substitutes for remdesivir. Remdesivir is cleaved inside cells to an active metabolite, referred to as GS441524. Therefore, GS441524 was tested as a possible substitute for remdesivir. As shown, while GS441524 exerted a smaller, yet significant, antiviral effect, it did not replace remdesivir in the REC combination (FIGS. 13 and 17). When optimized for maximal cytopathic effect, GS441524 failed to demonstrate criticality (FIGS. 15 and 19). Another potential viral RNA polymerase inhibitor, galidesivir, was also tested. As shown in FIG. 13, galidesivir is minimally potent on its own, but is enhanced be elacridar, suggesting that Galidesivir is also subject to cytoplasmatic export through ABCB1. Galidesivir was shown to somewhat enhance the effect of the REC combination (FIG. 14) with a contribution factor of 9.6, but failed to do so in another assay (FIG. 18). Overall, galidesivir was determined not to be a substitute for remdesivir in the REC combination. Another potential antiviral polymerase inhibitor, EIDD-2801, also failed as a substitute for remdesivir. EIDD-2801 is a prodrug, which was initially developed to treat influenza, and is converted to EIDD-1931, which is an active metabolite. Both EIDD-2801 (FIG. 13) and EIDD-1931 (FIG. 16) were tested. As shown, EIDD-1931 and EIDD-2038 had no effect on the REC combination and failed to substitute for remdesivir. Accordingly, remdesivir is uniquely operating as a functional viral polymerase inhibitor against the SARS-CoV2 RNA polymerase.

The specificity of remdesivir in the REC combination was shown to be remarkable. Various other ribonucleoside analogues (EIDD1931, EIDD2801, galidesivir, favipiravir, rimonavir) were tested, but none were able to substitute for remdesivir. The functional metabolite of remdesivir, GS441524, also failed to substitute for remdesivir. Therefore, cellular uptake of remdesivir was more effective than GS441524, and only the pro-drug was subjected to ABC-family export. Considering that remdesivir is currently administered clinically through intravenous (i.v.) infusion (mainly due to first-pass hepatic clearing) the enhanced potency of remdesivir attained by use of ABC-dual inhibition offers an opportunity for oral administration of the drug, as well as greater potency.

### Example 7: Identification of Additional Compounds that Enhance the Activity of the REC Combination

In our high-dimensional testing of compounds against SARS-CoV2 we explored approximately 250 unique, and known, anti-viral type drugs. In the initial screen, which encompassed 20 HD-DoE experiments, several emerging hits were identified. These hits were distributed across the library and, therefore, had not been evaluated against the REC combination. Accordingly, these hits were tested to identify additional compounds that could enhance the REC combination through possible quadruple synergism. None were found. The best emerging hits included JQ-1 (a BRD2/4 bromodomain inhibitor), ponatinib (a tyrosine kinase inhibitor), Z-VAD (an apoptosis inhibitor, targeting the Caspase apoptotic protein class), Q-VD-Oph (also an apoptosis inhibitor, targeting the Caspase apoptotic protein class). As shown in FIGS. 14 and 18, none of the compounds synergized with the REC combination. Arbidol, a possible inhibitor against the viral protease (and tested against in COVID19 clinically) also failed to synergize with the REC combination (FIG. 16). Other components that may plausibly be involved to alleviate viral replication and viral burden, such as zinc, vit-D3, and iron chelator, such as deferioxamine, failed to enhance the REC combination. Similarly, other drugs of interest, such as mefloquine, hydroxychloroquine, R428, nelfinavir, and dapivirine, all failed to enhance the REC combination. Accordingly, the REC combination is rare.

As demonstrated, the specificity of remdesivir in the REC combination is remarkable. Various other ribonucleoside analogues (EIDD1931, EIDD2801, galidesivir, favipiravir, rimonavir) were tested, but none were able to substitute for remdesivir. The functional metabolite of remdesivir, GS441524, also failed to substitute for remdesivir. Accordingly, the cellular uptake of remdesivir is more effective than GS441524 and only the pro-drug is subjected to ABC-family export. Considering that remdesivir is currently administered clinically through i.v. infusion mainly due to first-pass hepatic clearing, the enhanced potency of remdesivir attainable by use of ABC-dual inhibition makes it capable of being administered orally which, in turn, allows it to attain greater potency, and thus extend the use of the drug.

In addition to the data provided herein regarding the REC combination, additional empirical evidence is shown for multiple drugs currently being explored for efficacy against SARS-CoV2. The initial HD-DoE screen included two focus compounds—favipiravir, and hydroxychloroquine. Both drugs failed to emerge as efficacious, individually, and in combination with others, against SARS-CoV2 in the VERO6 EGFP assay, irbesartan, camostat, ritonavir, lopinavir, rimantadine, EIDD1931, mefloquine, arbidol, reviewed in (McKee et al. (2020) Candidate drugs against SARS-CoV-2 and COVID-19. Pharmacol. Res.), are all strong candidates for SARS-CoV2; however, this study did not provide evidence for efficacy. The antihelminth drug ivermectin, which is currently undergoing clinical testing in COVID-19 (e.g. NCT04351347, NCT04392713, NCT04360356), displayed individual, but limited potency against SARS-CoV2, but was unable to enhance the REC combination.

### Example 8: Determination of the IC50 of the REC Combination

A serial dilution assay was performed to determine the IC50 of the REC combination. Starting at 10 μM equimolar mixtures of the components of the REC combination, the viability of VERO-EGFP cells was determined. For all dilutions, extending to the lowest tested (at 50 nM) the REC combination exerted complete protection from the SARS-CoV2 virus (FIG. 19). As shown in FIG. 19, each drug was provided in equimolar concentration to others and applied to VERO-EGFP cells serially diluted from 10 uM to 50 nM. Two independent experiments are shown. At all concentrations tested, the REC combination exerted complete protection from SARS-CoV2 cytopathy. The EC50 was determined to be in the low nanomolar range, e.g., at least less than 71.8 nM, for inhibition of SARS-CoV2 cytopathy in VERO-EGFP cells.

### Example 9: Cell-Based Antiviral Assay of Huh7 Cells Infected with SARS-CoV-2

Cell Culture and Preparation of Virus Stock for Assays in Huh7 Cells

The human hepatoma cell line, Huh7, was maintained in Dulbecco's modified Eagle's medium (DMEM) supplemented with 10% fetal bovine serum (FBS), 2% HEPES 1M, 5 mL sodium bicarbonate 7.5%, 1% non-essential amino acids, and 1% Penicillin-Streptomycin 10,000 U/mL in a humidified 5% CO2 incubator at 37° C. Assay medium for producing virus stocks and antiviral testing was prepared by supplementing DMEM with 4% FBS, 2% HEPES 1M, 5 mL sodium bicarbonate 7.5% and 1% NEAA.

To quantify antiviral activity of remdesivir, combinations on Huh7 cells (a SARS-CoV-2 virus strain that produces sufficient cytopathic effect (CPE) on this cell line) was prepared as follows. Starting from passage 6 of the SARS-CoV-2 strain BetaCov/Belgium/GHB-03021/2020 (EPI ISL 40797612020-02-03; which was isolated from a Belgian patient returning from Wuhan in February 2020) three additional passages were completed on Huh7 cells. Cultures which showed the greatest CPE were selected. This resulted in a virus stock (passage 9) that confers full CPE on Huh7 (5.6×10\hat{ }4 TCID50/mL), as well as on Vero E6 cells (1.8×10\hat{ }7 TCID50/mL). The genotype of this virus stock shows four nucleotide changes as compared with the mother virus stock (P6).

Assays in Huh7 Cells

Remdesivir alone, and in combination with elacridar, was tested in antiviral assays with Huh7 cells to determine whether a synergistic benefit could be observed in the combination. The assay measures the extent to which these drugs interfere with the viral cytopathy of SARS-CoV-2 in host cells. Considering that viral load drives the clinical presentation and progression of viral-induced disease, interference with viral production in host cells provides significant clinical benefits. Huh7 is an immortalized cell line composed of epithelial-like cells derived originally from a human liver tumor. This line is susceptible to viral infection and supports viral replication, and thus provides a useful model of viral infection in human tissue.

The anti-viral effects of drug combinations were assayed in untreated, infected control cells, i.e., under conditions where the virus-induced cytopathic effect (CPE) was fully in effect. Huh7 cells were seeded in 96-well plates at a density of 6000 cells per well in assay medium. After overnight growth, cells were preincubated with candidate antiviral combinations for 2 hours prior to infection with multiplicity of infection (MOI) of 0.01 TCID50/cell. Cytopathic effect (CPE) was determined using MTS ((3-(4,5-dimethylthiazol-2-yl)-5-(3-carboxymethoxyphenyl)-2-(4-sulfophenyl)-2H-tetrazolium)) as an indicator of residual metabolic activity in cells 4 days post-infection. For this assay, an MTS/phenazine methosulphate (PMS) stock solution (2 mg/mL MTS and 46 μg/mL PMS in PBS at pH 6-6.5) was diluted 1/20 in MEM without phenol red. Medium was aspirated from wells of the test plates and 70 μL of MTS/PMS solution was added. After 0.5-1 hour incubation at 37° C. absorbance was measured at 498 nm. Cytotoxic effects caused by compound treatment alone were monitored in parallel plates containing mock-infected cells.

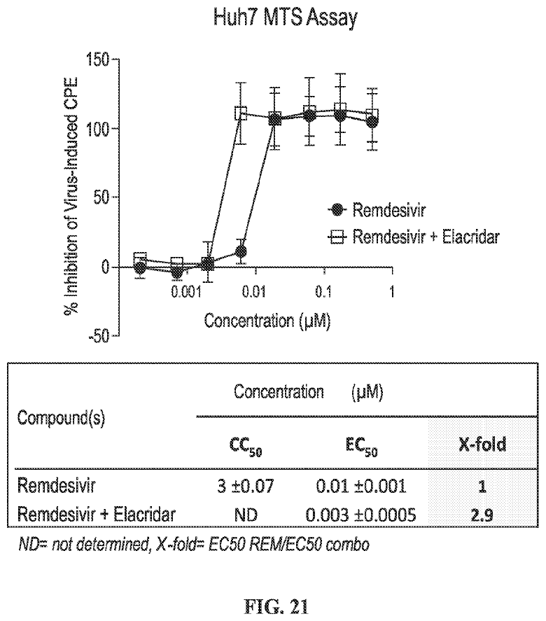

Remdesivir alone, and remdesivir in combination with elacridar, were tested for antiviral activity at a range of concentrations. Percent inhibition of virus-induced cytopathic effect was calculated as 100%×[A₄₉₈(treated, infected cells)−A₄₉₈(untreated, infected cells)]/[A₄₉₈(treated, mock-infected cells)−A₄₉₈(untreated, infected cells)], where A₄₉₈ was the absorbance measurement in the MTS assay. Compounds were tested at a range of concentrations using an 8-point dilution series with 3-fold serial dilutions starting at 0.5 μM. FIG. 21 displays the results. EC₅₀ values (50% effective concentration for CPE inhibition) were calculated based on the dose-response data and are summarized in FIG. 21.

Cytoxicity of remdesivir alone was determined for Huh7 cells using the same method at a higher range of remdesivir concentrations, but without introducing virus. The 50% cytotoxic concentration (CC₅₀) for remdesivir was 3±0.07 μM. Thus, the antiviral effect of remdesivir was measured in these studies at concentrations significantly below cytotoxic concentration.

A significant enhancement in the reduction of CPE was observed for the combination of remdesivir and elacridar in comparison with remdesivir alone. The ratios of EC₅₀ for remdesivir alone to EC₅₀ for remdesivir combinations is tabulated as ‘X-fold’ in FIG. 21. At the 0.0062 μM concentration level for remdesivir with elacridar, the cytopathic effect of the virus was completely abolished, whereas cells treated with remdesivir alone at this concentration were minimally protected from the effect of the virus. Accordingly, treatment with elacridar, a dual ABCB1/ABCG2 inhibitor, enhanced the antiviral effect of remdesivir.

Cell Culture and Preparation of Virus Stock for Assays in Vero E6 Cells

Vero E6 WT cells and Vero E6 GFP cells were maintained in DMEM supplemented with heat-inactivated 10% v/v fetal calf serum (FCS) and 500 μg/mL Geneticin and kept under 5% CO2 at 37° C. Assay medium for Vero E6 cells was DMEM supplemented with 2% v/v FCS. Virus stock was prepared as described above for assays in Huh7 cells.

Assays in Vero E6 Cells

Combinations of remdesivir, elacridar, and curcumin were also tested in cell-based antiviral assays with Vero E6 cells as the host cell. Both wild type cells (Vero E6 WT) and cells transformed to express green fluorescent protein (Vero E6-GFP) were used. Vero E6 is a cell line derived from kidney epithelial cells from African green monkey.

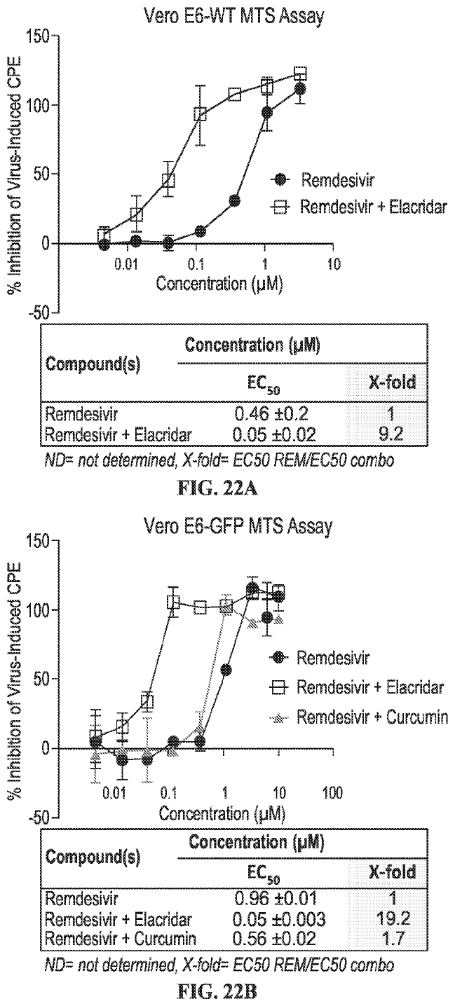

Cells were preincubated with candidate antiviral combinations for 2 hours prior to infection with SARS-CoV-2 virus (MOI=0.01). Three days post-infection, cytopathic effect (CPE) was determined using either MTS as described above. Compounds were assayed in the following combinations: (1) remdesivir alone, (2) curcumin alone, (3) elacridar alone, (4) remdesivir with elacridar, and (5) remdesivir with curcumin (Vero E6-GFP cells only). Percent inhibition of virus-induced cytopathic effect was calculated as for Huh7 cells. Compounds were tested at concentrations ranging from 0.0046 μM to 10 μM. FIGS. 22A and 22B displays the results. EC₅₀ values (50% effective concentration for CPE inhibition) were calculated based on the dose-response data and are summarized in FIGS. 22A and 22B.

In all the Vero E6 assays, a significant enhancement in the reduction of CPE was observed for the combination of remdesivir and elacridar in comparison with remdesivir alone. The addition of elacridar to remdesivir improves EC₅₀ for the antiviral effect by a factor of 9.2 (Vero E6 WT cells) to 19.2 (Vero E6 GFP cells). In addition, in Vero E6-GFP cells, the combination of remdesivir and curcumin exhibited a greater antiviral effect than remdesivir alone. The beneficial effect of adding curcumin is seen most clearly at the 1.11 μM treatment concentration. At this concentration, the cytopathic effect was eliminated in cells treated with remdesivir and curcumin. For cells treated with remdesivir only at the same concentration (i.e., 1.11 μM) the virus-induced cytopathic effect was 50-60% inhibited. Curcumin alone did not result in any significant antiviral effect in these assays (data not shown). Elacridar alone had a modest antiviral effect: EC₅₀ for % inhibition of virus-induced CPE was 1.07±0.5 μM in Vero E6-WT cells, and 2.16±0.5 μM in Vero E6-GFP cells. Therefore, the antiviral effect of the combinations was greater than one would expect if the drugs were acting together additively.

### Example 10: Virus Challenge Assays in Syrian Golden Hamster

Preclinical virus challenge assays in Syrian golden hamster were performed to test the synergistic effect of combining the antiviral drug remdesivir with tariquidar (a dual-specificity ABC-family inhibitor). The Syrian golden hamster model of SARS-CoV-2 infection is a valuable model system for evaluating therapeutic interventions for COVID-19 as the animals exhibit clinical signs of viral-induced morbidity and viral replication in relevant tissues, including lung, blood, and nasal washes.

Hamsters were infected intranasally with SARS-CoV-2 virus (2×10⁶ TCID₅₀ inoculum). Following infection, animals were treated twice daily on days 0-3 with vehicle, remdesivir alone (20 mg/kg, subcutaneous route (SC)), or remdesivir (20 mg/kg, SC) combined with other treatments, including tariquidar (5 mg/kg, SC). On day 4, animals were sacrificed, and lung tissue was harvested and analyzed to determine both the viral genome levels and the level of infectious virus in the lungs.

Viral genome levels and infectious virus levels were determined in the different experimental groups. Statistical comparisons between groups were made using a two-tailed Mann-Whitney test. Notably, the group treated with remdesivir and tariquidar showed improved anti-viral effect when compared to the group treated with remdesivir alone and the group treated with vehicle control. That is, a pronounced reduction of viral genome level was observed in individual animals in the remdesivir plus tariquidar group (data not shown).

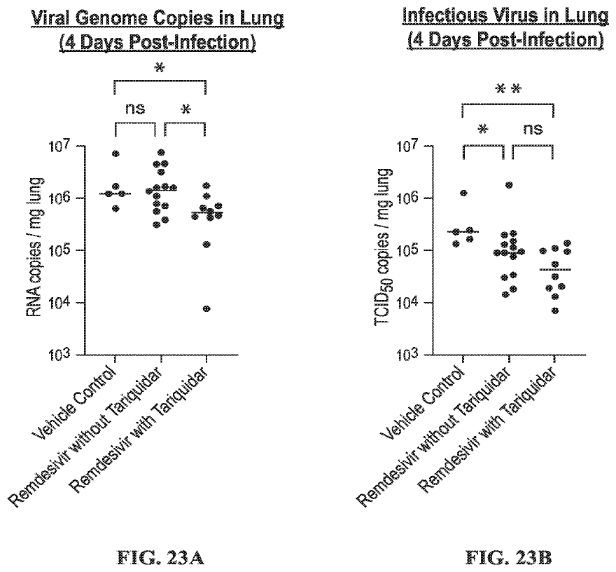

To further examine the combinatorial effect of remdesivir and tariquidar, the data were re-analyzed using a group pooling strategy. FIGS. 23A and 23B show the results of the viral challenge trial using the group pooling strategy. An enhanced anti-viral effect was observed in animals treated with remdesivir and tariquidar in combination. Statistical comparisons between groups were made using a two-tailed Mann-Whitney test. Viral genome levels in lung tissue were significantly reduced in the remdesivir with tariquidar group in comparison with both the remdesivir without tariquidar (P-value=0.0162) and vehicle control groups (P-value=0.028). For this endpoint, no significant difference was observed in comparing remdesivir without tariquidar group to the vehicle control group. Additionally, levels of infectious virus were reduced in the remdesivir with tariquidar group versus control to a greater level of statistical significance than the reduction seen for the remdesivir without tariquidar group versus control (P-value of 0.0013 for the former, P-value of 0.0185 for the latter).

Accordingly, administration of tariquidar (which alone would be expected to have no antiviral effect) enhanced the antiviral potency of remdesivir in lung tissue, and further demonstrated that the combination of tariquidar with remdesivir will lower the dose of remdesivir required for antiviral efficacy.

### EQUIVALENTS

Those skilled in the art will recognize or be able to ascertain, using no more than routine experimentation, many equivalents of the specific embodiments described herein described herein. Such equivalents are intended to be encompassed by the following claims.

We claim:

1. A method of treating a subject infected with a virus, or a subject at risk of infection with a virus, comprising administering to the subject an effective amount of a combination of compound A and compound B, or pharmacological analogues thereof, wherein:
compound A is a dual-specificity ABCB1/ABCG2 inhibitor and compound B is remdesivir, or a functional analog thereof, and
optionally, wherein the combination comprises compound C, wherein compound C is a NRF2 agonist,
wherein the subject is treated.

2. The method of claim 1, wherein compound A is elacridar, tariquidar, or zosuquidar, and/or wherein compound C is curcumin, or functional analogs thereof.

3. The method of claim 1 or 2, wherein
(a) compound A is administered in a dosage amount of about 20 mg to 300 mg;
(b) compound B is administered in a dosage amount of about 100 mg to about 200 mg; and
(c) if present, compound C is administered in a dosage amount of about 100 mg to about 1000 mg.

4. The method of any one of claims 1-3, comprising administering a combination of compound A, compound B, and compound C, or a pharmacological analogue thereof.

5. The method of any one of claims 1-4, wherein the virus belongs to the family coronaviridae, arenaviridae, or filoviridae.

6. The method of claim 5, wherein the virus is severe acute respiratory syndrome (SARS) coronavirus (SARS-CoV), Middle East respiratory syndrome (MERS) coronavinis (MERS-CoV), or SARS-CoV-2 (COVID-19).

7. The method of any one of claims 1-6, wherein compound A is administered once daily.

8. The method of any one of claims 1-7, wherein compound A is administered orally or by inhalation.

9. The method of any one of claims 1-7, wherein compound A is administered intravenously.

10. The method of any one of claims 1-9, wherein compound C is administered once daily in an amount of 500 mg or 25 mg/kg.

11. The method of any one of claims 1-10, wherein compound C is administered orally or by inhalation.

12. The method of any one of claims 1-10, wherein compound C is administered intravenously.

13. The method of any one of claims 1-12, wherein compound B is administered once daily in an amount of 200 mg or at a dose of 5 mg/kg.

14. The method of any one of claims 1-13, wherein compound B is administered orally or by inhalation.

15. The method of any one of claims 1-13, wherein compound B is administered intravenously.

16. The method of any one of claims 1-10, wherein the combination is administered orally, intravenously, or by inhalation.

17. The method of any one of claims 1-16, wherein the compounds are administered simultaneously (e.g., in a single formulation or concurrently as separate formulations).

18. The method of any one of claims 1-16, wherein the compounds are administered sequentially (e.g., as separate formulations).

19. The method of claim 18, wherein compound A is administered before compound B is administered.

20. The method of any one of claims 1-19 wherein the subject is not displaying SARS-CoV-2 and treatment is measured by prophylactic resistance against SARS-CoV2 infection.

21. The method of any one of claims 1-20, wherein the subject is identified as Sars-CoV-2 positive for virus RNA.

22. The method of any one of claims 1-21, wherein the subject is identified as Sars-CoV-2 positive for SARS-CoV2 antibodies.

23. The method of any one of claims 1-22, wherein the subject is diagnosed with bilateral pulmonary pneumonia.

24. The method of any one of claims 1-23, wherein the subject is diagnosed with SARS-CoV-2 related organ failures.

25. The method of any one of claims 1-24, wherein the SARS-CoV-2 viral infection is a latent infection.

26. The method of any one of claims 1-25, further comprising administration of an immunosuppressive therapy.

27. A composition for treating a subject infected with a virus, or a subject at risk of infection with a virus, comprising an effective amount of a combination of compound A and compound B, or pharmacological analogues thereof, wherein:
compound A is a dual-specificity ABCB1/ABCG2 inhibitor and compound B is remdesivir, or a functional analog thereof, and
optionally, wherein the combination comprises compound C, wherein compound C is a NRF2 agonist.

28. The composition of claim 27, wherein compound A is elacridar, tariquidar, or zosuquidar, and/or wherein compound C is curcumin, or functional analogs thereof.

29. The composition of claim 27 or 28, wherein
(a) compound A is in a dosage amount of about 20 mg to 300 mg:
(b) compound B is in a dosage amount of about 20 mg to about 200 mg; and
(c) if present, compound C is in a dosage amount of about 100 mg to about 1000 mg.

30. The composition of any one of claims 27-29, wherein compound C is curcumin.

31. The composition of any one of claims 27-30, wherein the virus belongs to the family coronaviridae, arenaviridae, or filoviridae.

32. The composition of any one of claims 27-31, wherein the virus is severe acute respiratory syndrome (SARS) coronavirus (SARS-CoV), Middle East respiratory syndrome (MERS) coronavirus (MERS-CoV), or SARS-CoV-2 (COVID-19).

33. A composition for treating a subject infected with severe acute respiratory syndrome coronavirus 2 (SARS-CoV-2), or a subject at risk of infection with SARS-Co-V-2, comprising a combination of compound A, compound B, and compound C, wherein
(a) compound A is in a dosage amount of about 50 mg to 300 mg and is elacridar or tariquidar,
(b) compound B is in a dosage amount of about 100 mg to about 200 and is remdesivir, and
(c) compound C is in a dosage amount of about 100 mg to about 1000 mg and is curcumin.

34. The composition of any one of claims 27-33, wherein the composition is formulated for intravenous administration, oral administration, inhalation, or intra-muscular administration.

35. A method of inhibiting infection of a cell by severe acute respiratory syndrome coronavirus 2 (SARS-CoV-2), comprising contacting the cell with the composition of any one of claims 27-35.

36. A kit comprising a combination of compound A and compound B, or pharmacological analogues thereof, and instructions for use, wherein,
compound A is a dual-specificity ABCB1/ABCG2 inhibitor and compound B is remdesivir, or a functional analog thereof, and
optionally, wherein the combination comprises compound C, wherein compound C is a NRF2 agonist.

37. The kit of claim 36, wherein compound A is elacridar, tariquidar, or zosuquidar, and/or wherein compound C is curcumin, or functional analogs thereof.

38. The kit of claim 36 or 37, wherein
(a) compound A is in a dosage amount of about 20 mg to 300 mg;
(b) compound B is in a dosage amount of about 20 mg to about 200 mg; and
(c) if present, compound C is in a dosage amount of about 100 mg to about 1000 mg.
